(19) United States
(12) Patent Application Publication
(10) Pub. No.: US 2019/0002399 A1
(43) Pub. Date: Jan. 3, 2019

(54) OLIGOMER CONJUGATES OF LIDOCAINE AND ITS DERIVATIVES

(71) Applicant: Nektar Therapeutics, San Francisco, CA (US)

(72) Inventors: Jennifer Riggs-Sauthier, Huntsville, AL (US); Bo-Liang Deng, San Ramon, CA (US)

(21) Appl. No.: 16/115,310

(22) Filed: Aug. 28, 2018

Related U.S. Application Data
(62) Division of application No. 12/682,778, filed on Jun. 18, 2010, now abandoned.
(60) Provisional application No. 60/999,570, filed on Oct. 19, 2007.

(51) Int. Cl. C07C 237/04; A61K 47/55; C07C 217/18; A61K 47/60

(52) U.S. Cl. CPC C07C 237/04; A61K 47/55; A61K 47/60; C07C 217/18

(57) ABSTRACT

The invention provides small molecule drugs that are chemically modified by covalent attachment of a water-soluble oligomer.

### CROSS REFERENCE TO RELATED APPLICATION

This application is a divisional of U.S. patent application Ser. No. 12/682,778, filed Jun. 18, 2010, which is a 35 U.S.C. § 371 application of International Application No. PCT/US2008/011880, filed Oct. 17, 2008, designating the United States, which claims the benefit of priority under 35 U.S.C. § 119(e) to U.S. Provisional Patent Application No. 60/999,570, filed Oct. 19, 2007, all of which are hereby incorporated by reference in their entireties.

### FIELD OF THE INVENTION

This invention comprises (among other things) chemically modified lidocaine and its derivatives that possess certain advantages over versions lacking the chemical modification. The chemically modified versions described herein relate to and/or have application(s) in (among others) the fields of drug discovery, pharmacotherapy, physiology, organic chemistry and polymer chemistry.

### BACKGROUND OF THE INVENTION

Lidocaine and its derivatives (e.g., tocainide, mexiletine) are typically classified as Class IB antiarrhythmic drugs. The mechanism of action for this class of drugs is believed to be related to their ability to block sodium channels which results in suppressed cardiac electrical activity of abnormally polarized cardiac tissues. Lidocaine and its derivatives have also been used as analgesics.

Some drugs in the class, and lidocaine in particular, are extensively metabolized via first-pass hepatic metabolism, thereby requiring parenteral administration. In addition, administration of drugs in this class (regardless of the route of administration) is often associated with neurologically focused side effects, such as paresthesias, tremor, blurred vision, lethargy, light-headedness, hearing disturbances, slurred speech and convulsions.

One approach for avoiding the problems associated with extensive first pass metabolism is to administer the drug via intravenous administration. Indeed, lidocaine is typically administered by the intravenous route to avoid extensive first pass metabolism. Intravenous administration, however, requires the use of trained clinical personnel, which may be inconvenient or impractical.

In addition to solving the problems associated with intravenous administration, it would be desirable to have a compound that has the activity of lidocaine (or its derivatives) without the neurologically focused side effects.

The present invention seeks to address these and other needs in the art.

### SUMMARY OF THE INVENTION

In one or more embodiments of the invention, a compound is provided, the compound comprising a lidocaine residue covalently attached via a stable or degradable linkage to a water-soluble, non-peptidic oligomer.

Exemplary compounds of the invention include those having the following structure:

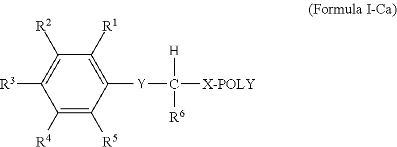

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

Y is selected from the group consisting of —O—, —O—CH₂—, —CH₂—O—, —NH—, —NHC(O)—, —C(O)NH—, —CH₂— and —CH₂—CH₂—;

X is a spacer moiety; and

POLY is a water-soluble, non-peptidic oligomer.

Additional exemplary compounds of the invention include those having the following structure:

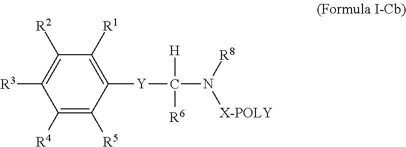

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁸ is hydrogen or lower alkyl;

Y is selected from the group consisting of —O—, —O—CH₂—, —CH₂—O—, —NH—, —NHC(O)—, —C(O)NH—, —CH₂— and —CH₂—CH₂—;

X is a spacer moiety; and

POLY is a water-soluble, non-peptidic oligomer.

The “lidocaine residue” is a compound having a structure of lidocaine or a derivative thereof that is altered by the presence of one or more bonds, which bonds serve to attach (either directly or indirectly) one or more water-soluble, non-peptidic oligomers. In this regard, any compound having lidocaine activity can be used. Exemplary lidocaine derivatives have a structure encompassed by the structure defined herein as Formula I, as follows:

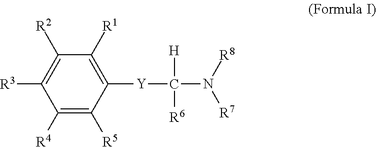

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁷ is hydrogen or lower alkyl;

R⁸ is hydrogen or lower alkyl; and

Y is selected from the group consisting of —O—, —O—CH₂—, —CH₂—O—, —NH—, —NHC(O)—, —C(O)NH—, —CH₂— and —CH₂—CH₂—.

In one or more embodiments of the invention, a composition is provided, the composition comprising a compound comprising a residue of lidocaine or a derivative thereof covalently attached via a stable or degradable linkage to a water-soluble, non-peptidic oligomer, and optionally, a pharmaceutically acceptable excipient.

In one or more embodiments of the invention, a dosage form is provided, the dosage form comprising a compound comprising a residue of lidocaine or a derivative thereof covalently attached via a stable or degradable linkage to a water-soluble, non-peptidic oligomer, wherein the compound is present in a dosage form.

In one or more embodiments of the invention, a method is provided, the method comprising covalently attaching a water-soluble, non-peptidic oligomer to lidocaine or a derivative thereof.

In one or more embodiments of the invention, a method is provided, the method comprising administering a compound comprising a residue of lidocaine or a derivative thereof covalently attached via a stable or degradable linkage to a water-soluble, non-peptidic oligomer.

These and other objects, aspects, embodiments and features of the invention will become more fully apparent to one of ordinary skill in the art when read in conjunction with the following detailed description.

### BRIEF DESCRIPTION OF THE FIGURES

FIG. 1A and FIG. 1B are graphs of the “lidocaine series” of results obtained in accordance with the description provided in Example 3.

FIG. 2A and FIG. 2B are graphs of the “mexiletine series” of results obtained in accordance with the procedure set forth in Example 3.

FIG. 3 is a graph of the “lidocaine series” of acid writhing data obtained in accordance with the procedure set forth in Example 4.

FIG. 4 is a graph of the “mexiletine series” of acid writhing data obtained in accordance with the procedure set forth in Example 4.

### DETAILED DESCRIPTION OF THE INVENTION

As used in this specification, the singular forms “a,” “an,” and “the” include plural referents unless the context clearly dictates otherwise.

In describing and claiming the present invention, the following terminology will be used in accordance with the definitions described below.

“Water soluble, non-peptidic oligomer” indicates an oligomer that is at least 35% (by weight) soluble, preferably greater than 70% (by weight), and more preferably greater than 95% (by weight) soluble, in water at room temperature. Typically, an unfiltered aqueous preparation of a “water-soluble” oligomer transmits at least 75%, more preferably at least 95%, of the amount of light transmitted by the same solution after filtering. It is most preferred, however, that the water-soluble oligomer is at least 95% (by weight) soluble in water or completely soluble in water. With respect to being “non-peptidic,” an oligomer is non-peptidic when it has less than 35% (by weight) of amino acid residues.

The terms “monomer,” “monomeric subunit” and “monomeric unit” are used interchangeably herein and refer to one of the basic structural units of a polymer or oligomer. In the case of a homo-oligomer, a single repeating structural unit forms the oligomer. In the case of a co-oligomer, two or more structural units are repeated—either in a pattern or randomly—to form the oligomer. Preferred oligomers used in connection with present the invention are homo-oligomers. The water-soluble, non-peptidic oligomer typically comprises one or more monomers serially attached to form a chain of monomers. The oligomer can be formed from a single monomer type (i.e., is homo-oligomeric) or two or three monomer types (i.e., is co-oligomeric).

An “oligomer” is a molecule possessing from about 1 to about 30 monomers. Specific oligomers for use in the invention include those having a variety of geometries such as linear, branched, or forked, to be described in greater detail below.

“PEG” or “polyethylene glycol,” as used herein, is meant to encompass any water-soluble poly(ethylene oxide). Unless otherwise indicated, a “PEG oligomer” is one in which substantially all (preferably all) monomeric subunits are ethylene oxide subunits, though the oligomer may contain distinct end capping moieties or functional groups, e.g., for conjugation. PEG oligomers for use in the present invention will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG oligomers, the variable (n) ranges from 1 to 30, and the terminal groups and architecture of the overall PEG can vary. When PEG further comprises a functional group, A, for linking to, e.g., a small molecule drug, the functional group when covalently attached to a PEG oligomer does not result in formation of (i) an oxygen-oxygen bond (—O—O—, a peroxide linkage), or (ii) a nitrogen-oxygen bond (N—O, O—N).

The terms “end-capped” or “terminally capped” are interchangeably used herein to refer to a terminal or endpoint of a polymer having an end-capping moiety. Typically, although not necessarily, the end-capping moiety comprises a hydroxy or C₁₋₂₀ alkoxy group. Thus, examples of end-capping moieties include alkoxy (e.g., methoxy, ethoxy and benzyloxy), as well as aryl, heteroaryl, cyclo, heterocyclo, and the like. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) of interest to which the polymer is coupled, can be determined by using a suitable detector. Such labels include, without limitation, fluorescers, chemiluminescers, moieties used in enzyme labeling, colorimetric moieties (e.g., dyes), metal ions, radioactive moieties, and the like. Suitable detectors include photometers, films, spectrometers, and the like.

“Branched,” in reference to the geometry or overall structure of an oligomer, refers to an oligomer having two or more polymer “arms” extending from a branch point.

“Forked,” in reference to the geometry or overall structure of an oligomer, refers to an oligomer having two or more functional groups (typically through one or more atoms) extending from a branch point.

A “branch point” refers to a bifurcation point comprising one or more atoms at which an oligomer branches or forks from a linear structure into one or more additional arms.

The term “reactive” or “activated” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive,” with reference to a functional group present on a molecule in a reaction mixture, indicates that the group remains largely intact under conditions that are effective to produce a desired reaction in the reaction mixture.

A “protecting group” is a moiety that prevents or blocks reaction of a particular chemically reactive functional group in a molecule under certain reaction conditions. The protecting group may vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein.

A functional group in “protected form” refers to a functional group bearing a protecting group. As used herein, the term “functional group” or any synonym thereof encompasses protected forms thereof.

A “physiologically cleavable” or “hydrolyzable” or “degradable” bond is a relatively labile bond that reacts with water (i.e., is hydrolyzed) under physiological conditions. The tendency of a bond to hydrolyze in water may depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, thiolesters, and carbonates.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages include but are not limited to the following: carbon-carbon bonds (e.g., in aliphatic chains), ethers, amides, urethanes, amines, and the like. Generally, a stable linkage is one that exhibits a rate of hydrolysis of less than about 1-2% per day under physiological conditions. Hydrolysis rates of representative chemical bonds can be found in most standard chemistry textbooks.

“Substantially” or “essentially” means nearly totally or completely, for instance, 95% or greater, more preferably 97% or greater, still more preferably 98% or greater, even more preferably 99% or greater, yet still more preferably 99.9% or greater, with 99.99% or greater being most preferred of some given quantity.

“Monodisperse” refers to an oligomer composition wherein substantially all of the oligomers in the composition have a well-defined, single molecular weight and defined number of monomers, as determined by chromatography or mass spectrometry. Monodisperse oligomer compositions are in one sense pure, that is, substantially having a single and definable number (as a whole number) of monomers rather than a large distribution. A monodisperse oligomer composition possesses a MW/Mn value of 1.0005 or less, and more preferably, a MW/Mn value of 1.0000. By extension, a composition comprised of monodisperse conjugates means that substantially all oligomers of all conjugates in the composition have a single and definable number (as a whole number) of monomers rather than a large distribution and would possess a MW/Mn value of 1.0005, and more preferably, a MW/Mn value of 1.0000 if the oligomer were not attached to moiety derived from a small molecule drug. A composition comprised of monodisperse conjugates may, however, include one or more nonconjugate substances such as solvents, reagents, excipients, and so forth.

“Bimodal,” in reference to an oligomer composition, refers to an oligomer composition wherein substantially all oligomers in the composition have one of two definable and different numbers (as whole numbers) of monomers rather than large a distribution, and whose distribution of molecular weights, when plotted as a number fraction versus molecular weight, appears as two separate identifiable peaks. Preferably, for a bimodal oligomer composition as described herein, each peak is generally symmetric about its mean, although the size of the two peaks may differ. Ideally, the polydispersity index of each peak in the bimodal distribution, Mw/Mn, is 1.01 or less, more preferably 1.001 or less, and even more preferably 1.0005 or less, and most preferably a MW/Mn value of 1.0000. By extension, a composition comprised of bimodal conjugates means that substantially all oligomers of all conjugates in the composition have one of two definable and different numbers (as whole numbers) of monomers rather than a large distribution and would possess a MW/Mn value of 1.01 or less, more preferably 1.001 or less and even more preferably 1.0005 or less, and most preferably a MW/Mn value of 1.0000 if the oligomer were not attached to the moiety derived from a small molecule drug. A composition comprised of bimodal conjugates may, however, include one or more nonconjugate substances such as solvents, reagents, excipients, and so forth.

“Lidocaine and derivatives thereof” refer to an organic, inorganic, or organometallic small molecule drugs having a molecular weight of less than about 1000 Daltons and having some degree of lidocaine activity, such as the ability to stabilize the neuronal membrane by inhibiting the ionic fluxes required for the initiation and conduction of impulses.

A “biological membrane” is any membrane made of cells or tissues that serves as a barrier to at least some foreign entities or otherwise undesirable materials. As used herein a “biological membrane” includes those membranes that are associated with physiological protective barriers including, for example: the blood-brain barrier (BBB); the blood-cerebrospinal fluid barrier; the blood-placental barrier; the blood-milk barrier; the blood-testes barrier; and mucosal barriers including the vaginal mucosa, urethral mucosa, anal mucosa, buccal mucosa, sublingual mucosa and rectal mucosa. Unless the context clearly dictates otherwise, the term “biological membrane” does not include those membranes associated with the middle gastro-intestinal tract (e.g., stomach and small intestines).

A “biological membrane crossing rate,” provides a measure of a compound's ability to cross a biological membrane, such as the membrane associated with the blood-brain barrier (“BBB”). A variety of methods may be used to assess transport of a molecule across any given biological membrane. Methods to assess the biological membrane crossing rate associated with any given biological barrier (e.g., the blood-cerebrospinal fluid barrier, the blood-placental barrier, the blood-milk barrier, the intestinal barrier, and so forth), are known, described herein and/or in the relevant literature, and/or may be determined by one of ordinary skill in the art.

A “reduced rate of metabolism” refers to a measurable reduction in the rate of metabolism of a water-soluble oligomer-small molecule drug conjugate as compared to the rate of metabolism of the small molecule drug not attached to the water-soluble oligomer (i.e., the small molecule drug itself) or a reference standard material. In the special case of “reduced first pass rate of metabolism,” the same “reduced rate of metabolism” is required except that the small molecule drug (or reference standard material) and the corresponding conjugate are administered orally. Orally administered drugs are absorbed from the gastro-intestinal tract into the portal circulation and may pass through the liver prior to reaching the systemic circulation. Because the liver is the primary site of drug metabolism or biotransformation, a substantial amount of drug may be metabolized before it reaches the systemic circulation. The degree of first pass metabolism, and thus, any reduction thereof, can be measured by a number of different approaches. For instance, animal blood samples can be collected at timed intervals and the plasma or serum analyzed by liquid chromatography/mass spectrometry for metabolite levels. Other techniques for measuring a “reduced rate of metabolism” associated with the first pass metabolism and other metabolic processes are known, described herein and/or in the relevant literature, and/or can be determined by one of ordinary skill in the art. Preferably, a conjugate of the invention can provide a reduced rate of metabolism reduction satisfying at least one of the following values: at least about 30%; at least about 40%; at least about 50%; at least about 60%; at least about 70%; at least about 80%; and at least about 90%. A compound (such as a small molecule drug or conjugate thereof) that is “orally bioavailable” is one that preferably possesses a bioavailability when administered orally of greater than 25%, and preferably greater than 70%, where a compound's bioavailability is the fraction of administered drug that reaches the systemic circulation in unmetabolized form.

“Alkyl” refers to a hydrocarbon chain, ranging from about 1 to 20 atoms in length. Such hydrocarbon chains are preferably but not necessarily saturated and may be branched or straight chain. Exemplary alkyl groups include methyl, ethyl, propyl, butyl, pentyl, 2-methylbutyl, 2-ethylpropyl, 3-methylpentyl, and the like. As used herein, “alkyl” includes cycloalkyl when three or more carbon atoms are referenced.

“Lower alkyl” refers to an alkyl group containing from 1 to 6 carbon atoms, and may be straight chain or branched, as exemplified by methyl, ethyl, n-butyl, i-butyl, t-butyl.

“Non-interfering substituents” are those groups that, when present in a molecule, are typically non-reactive with other functional groups contained within the molecule.

“Alkoxy” refers to an —O—R group, wherein R is alkyl or substituted alkyl, preferably C₁-C₂₀ alkyl (e.g., methoxy, ethoxy, propyloxy, benzyl, etc.), preferably C₁-C₇.

“Pharmaceutically acceptable excipient” or “pharmaceutically acceptable carrier” refers to component that may be included in the compositions of the invention and causing no significant adverse toxicological effects to a patient.

The term “aryl” means an aromatic group having up to 14 carbon atoms. Aryl groups include phenyl, naphthyl, biphenyl, phenanthrenyl, naphthacenyl, and the like. “Substituted phenyl” and “substituted aryl” denote a phenyl group and aryl group, respectively, substituted with one, two, three, four or five (e.g. 1-2, 1-3 or 1-4 substituents) chosen from halo (F, Cl, Br, I), hydroxy, hydroxy, cyano, nitro, alkyl (e.g., C₁₋₆ alkyl), alkoxy (e.g., C₁₋₆ alkoxy), benzyloxy, carboxy, aryl, and so forth.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g., CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S).

“Pharmacologically effective amount,” “physiologically effective amount,” and “therapeutically effective amount” are used interchangeably herein to mean the amount of a water-soluble oligomer-small molecule drug conjugate present in a composition that is needed to provide a desired level of active agent and/or conjugate in the bloodstream or in the target tissue. The precise amount may depend upon numerous factors, e.g., the particular active agent, the components and physical characteristics of the composition, intended patient population, patient considerations, and may readily be determined by one skilled in the art, based upon the information provided herein and available in the relevant literature.

A “difunctional” oligomer is an oligomer having two functional groups contained therein, typically at its termini. When the functional groups are the same, the oligomer is said to be homodifunctional. When the functional groups are different, the oligomer is said to be heterobifunctional.

A basic reactant or an acidic reactant described herein include neutral, charged, and any corresponding salt forms thereof.

The term “patient,” refers to a living organism suffering from or prone to a condition that can be prevented or treated by administration of a conjugate as described herein, and includes both humans and animals.

“Optional” or “optionally” means that the subsequently described circumstance may but need not necessarily occur, so that the description includes instances where the circumstance occurs and instances where it does not.

As indicated above, the present invention is directed to (among other things) a compound comprising a residue of lidocaine or a derivative thereof covalently attached via a stable or degradable linkage to a water-soluble, non-peptidic oligomer.

In one or more embodiments of the invention, a compound is provided, the compound comprising a residue of lidocaine or a derivative thereof covalently attached via a stable or degradable linkage to a water-soluble, non-peptidic oligomer, wherein the lidocaine or derivative thereof has a structure encompassed by the following formula:

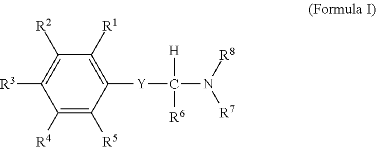

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁷ is hydrogen or lower alkyl;

R⁸ is hydrogen or lower alkyl; and

Y is selected from the group consisting of —O—, —O—CH₂—, —CH₂—O—, —NH—, —NHC(O)—, —C(O)NH—, —CH₂— and —CH₂—CH₂—.

The small molecule to which the water-soluble, nonpeptidic oligomer is attached can be lidocaine or a derivative thereof. Specific examples of which include lidocaine, tocainide and mexiletine.

It is believed that an advantage of the compounds of the present invention is their ability to retain some degree of lidocaine activity while also exhibiting a decrease in metabolism. Although not wishing to be bound by theory, it is believed that the residue of lidocaine and derivatives thereof—and oligomer-containing compounds described herein—(in contrast to the oligomer-free “original” lidocaine or derivative structure thereof) are not metabolized as readily because the oligomer serves to reduce the overall affinity of the compound to substrates that can metabolize lidocaine or its derivatives. In addition (and again, not wishing to be bound by theory), the extra size introduced by the oligomer—in contrast to the oligomer-free “original” lidocaine or derivative structure thereof—reduces the ability of the compound to cross the blood-brain barrier. Even should the linkage between the residue of lidocaine or derivative thereof and the oligomer be degradable, the compound still offers advantages (such as avoiding first-pass metabolism upon initial absorption).

Use of discrete oligomers (e.g., from a monodisperse or bimodal composition of oligomers, in contrast to relatively impure compositions) to form oligomer-containing compounds can advantageously alter certain properties associated with the corresponding small molecule drug. For instance, a compound of the invention, when administered by any of a number of suitable administration routes, such as parenteral, oral, transdermal, buccal, pulmonary, or nasal, exhibits reduced penetration across the blood-brain barrier. It is preferred that the compounds of the invention exhibit slowed, minimal or effectively no crossing of the blood-brain barrier, while still crossing the gastro-intestinal (GI) walls and into the systemic circulation if oral delivery is intended. Moreover, the compounds of the invention maintain a degree of bioactivity as well as bioavailability in comparison to the bioactivity and bioavailability of the compound free of all oligomers.

With respect to the blood-brain barrier (“BBB”), this barrier restricts the transport of drugs from the blood to the brain. This barrier consists of a continuous layer of unique endothelial cells joined by tight junctions. The cerebral capillaries, which comprise more than 95% of the total surface area of the BBB, represent the principal route for the entry of most solutes and drugs into the central nervous system.

For compounds whose degree of blood-brain barrier crossing ability is not readily known, such ability can be determined using a suitable animal model such as an in situ rat brain perfusion (“RBP”) model as described herein. Briefly, the RBP technique involves cannulation of the carotid artery followed by perfusion with a compound solution under controlled conditions, followed by a wash out phase to remove compound remaining in the vascular space. (Such analyses can be conducted, for example, by contract research organizations such as Absorption Systems, Exton, Pa.). In one example of the RBP model, a cannula is placed in the left carotid artery and the side branches are tied off. A physiologic buffer containing the analyte (typically but not necessarily at a 5 micromolar concentration level) is perfused at a flow rate of about 10 mL/minute in a single pass perfusion experiment. After 30 seconds, the perfusion is stopped and the brain vascular contents are washed out with compound-free buffer for an additional 30 seconds. The brain tissue is then removed and analyzed for compound concentrations via liquid chromatograph with tandem mass spectrometry detection (LC/MS/MS). Alternatively, blood-brain barrier permeability can be estimated based upon a calculation of the compound's molecular polar surface area (“PSA”), which is defined as the sum of surface contributions of polar atoms (usually oxygens, nitrogens and attached hydrogens) in a molecule. The PSA has been shown to correlate with compound transport properties such as blood-brain barrier transport. Methods for determining a compound's PSA can be found, e.g., in, Ertl, P., et al., J. Med. Chem. 2000, 43, 3714-3717; and Kelder, J., et al., Pharm. Res. 1999, 16, 1514-1519.

With respect to the blood-brain barrier, the water-soluble, non-peptidic oligomer-small molecule drug compound exhibits a blood-brain barrier crossing rate that is reduced as compared to the crossing rate of the small molecule drug not attached to the water-soluble, non-peptidic oligomer. Preferred exemplary reductions in blood-brain barrier crossing rates for the compounds described herein include reductions of; at least about 30%; at least about 40%; at least about 50%; at least about 60%; at least about 70%; at least about 80%; or at least about 90%, when compared to the blood-brain barrier crossing rate of the small molecule drug not attached to the water-soluble oligomer. A preferred reduction in the blood-brain barrier crossing rate for a compound of the invention is at least about 20%.

As indicated above, the compounds of the invention include a residue of lidocaine or a derivative thereof. Assays for determining whether a given compound (regardless of whether the compound includes a water-soluble, non-peptidic oligomer or not) has lidocaine activity are described infra.

Lidocaine as well as lidocaine derivatives are encompassed by the following formula:

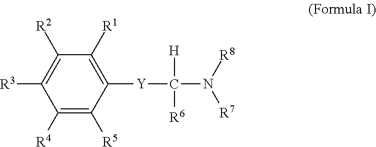

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁷ is hydrogen or lower alkyl;

R⁸ is hydrogen or lower alkyl; and

Y is selected from the group consisting of —O—, —O—CH₂—, —CH₂—O—, —NH—, —NHC(O)—, —C(O)NH—, —CH₂— and —CH₂—CH₂—.

In one or more embodiments, the lidocaine derivative is encompassed by the following structure:

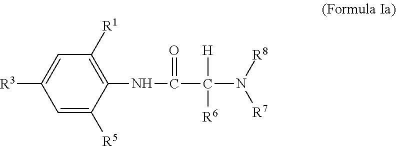

wherein: R¹ is hydrogen or lower alkyl (preferably methyl); R³ is hydrogen or lower alkyl (preferably hydrogen); R⁵ is hydrogen or lower alkyl (preferably methyl); R⁶ is hydrogen or lower alkyl (preferably methyl); R⁷ is hydrogen or lower alkyl; and R⁸ is hydrogen or lower alkyl. In one or more embodiments, the small molecule is lidocaine, which has the following structure:

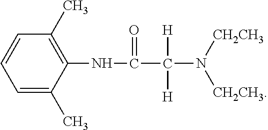 [SMILES: CCN(CC)CC(=O)Nc1c(C)cccc1C]

Compounds encompassed by Formula Ia can be prepared according to known methods. See, for example, U.S. Pat. No. 2,441,498 for a description of one or more methods for preparing compounds encompassed by or related to compounds having structures encompassed by Formula Ia.

In one or more embodiments, the lidocaine derivative is encompassed by the following structure:

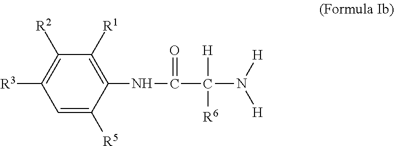

wherein: R¹ is hydrogen or lower alkyl (preferably methyl); R² is hydrogen or lower alkyl (preferably hydrogen); R³ is hydrogen or lower alkyl (preferably hydrogen); R⁵ is hydrogen or lower alkyl (preferably methyl); and R⁶ is hydrogen or lower alkyl (preferably methyl). In one or more embodiments, the lidocaine derivative is tocainide, which has the following structure:

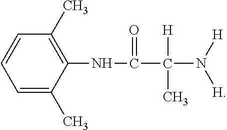

Lidocaine derivatives encompassed by Formula Ib can be prepared according to known methods. See, for example, U.S. Pat. No. 4,218,477 for a description of one or more methods for preparing lidocaine derivatives encompassed by or related to compounds having structures encompassed by Formula Ib.

In one or more embodiments, the lidocaine derivative is encompassed by the following structure:

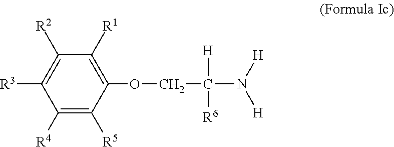

wherein: R¹ is hydrogen or lower alkyl (preferably methyl); R² is hydrogen or lower alkyl (preferably hydrogen); R³ is hydrogen or lower alkyl (preferably hydrogen); R⁴ is hydrogen or lower alkyl (preferably hydrogen); R⁵ is hydrogen or lower alkyl (preferably methyl); and R⁶ is hydrogen or lower alkyl (preferably methyl). In one or more embodiments, the lidocaine derivative is mexiletine, which has the following structure:

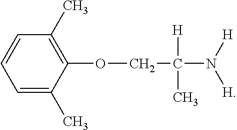

Lidocaine derivatives encompassed by Formula Ic can be prepared according to known methods. See, for example, U.S. Pat. No. 3,659,019 for a description of one or more methods for preparing lidocaine derivatives encompassed by or related to compounds having structures encompassed by Formula Ic.

In some instances, lidocaine and derivatives thereof can be obtained from commercial sources. In addition, lidocaine and derivatives thereof can be obtained through chemical synthesis. Examples of lidocaine and derivatives thereof as well as synthetic approaches for preparing the same are described in the literature and in, for example, U.S. Pat. Nos. 3,659,019, 4,218,477 and 2,441,498.

Each of these (and other) lidocaine derivatives can be covalently attached (either directly or through one or more atoms) to a water-soluble and non-peptidic oligomer.

Small molecule drugs useful in the invention generally have a molecular weight of less than 1000 Da. Exemplary molecular weights of small molecule drugs include molecular weights of: less than about 950; less than about 900; less than about 850; less than about 800; less than about 750; less than about 700; less than about 650; less than about 600; less than about 550; less than about 500; less than about 450; less than about 400; less than about 350; and less than about 300.

The small molecule drug used in the invention, if chiral, may be in a racemic mixture, or an optically active form, for example, a single optically active enantiomer, or any combination or ratio of enantiomers (i.e., scalemic mixture). In addition, the small molecule drug may possess one or more geometric isomers. With respect to geometric isomers, a composition can comprise a single geometric isomer or a mixture of two or more geometric isomers. A small molecule drug for use in the present invention can be in its customary active form, or may possess some degree of modification. For example, a small molecule drug may have a targeting agent, tag, or transporter attached thereto, prior to or after covalent attachment of an oligomer. Alternatively, the small molecule drug may possess a lipophilic moiety attached thereto, such as a phospholipid (e.g., distearoylphosphatidylethanolamine or “DSPE,” dipalmitoylphosphatidylethanolamine or “DPPE,” and so forth) or a small fatty acid. In some instances, however, it is preferred that the small molecule drug moiety does not include attachment to a lipophilic moiety.

Lidocaine and its derivatives for coupling to a water-soluble, non-peptidic oligomer possesses a free hydroxyl, carboxyl, thio, amino group, or the like (i.e., “handle”) suitable for covalent attachment to the oligomer. In addition, lidocaine or a derivative thereof can be modified by introduction of a reactive group, preferably by conversion of one of its existing functional groups to a functional group suitable for formation of a stable covalent linkage between the oligomer and the drug.

Accordingly, each oligomer is composed of up to three different monomer types selected from the group consisting of: alkylene oxide, such as ethylene oxide or propylene oxide; olefinic alcohol, such as vinyl alcohol, 1-propenol or 2-propenol; vinyl pyrrolidone; hydroxyalkyl methacrylamide or hydroxyalkyl methacrylate, where alkyl is preferably methyl; α-hydroxy acid, such as lactic acid or glycolic acid; phosphazene, oxazoline, amino acids, carbohydrates such as monosaccharides, saccharide or mannitol; and N-acryloylmorpholine. Preferred monomer types include alkylene oxide, olefinic alcohol, hydroxyalkyl methacrylamide or methacrylate, N-acryloylmorpholine, and α-hydroxy acid. Preferably, each oligomer is, independently, a co-oligomer of two monomer types selected from this group, or, more preferably, is a homo-oligomer of one monomer type selected from this group.

The two monomer types in a co-oligomer may be of the same monomer type, for example, two alkylene oxides, such as ethylene oxide and propylene oxide. Preferably, the oligomer is a homo-oligomer of ethylene oxide. Usually, although not necessarily, the terminus (or termini) of the oligomer that is not covalently attached to a small molecule drug is capped to render it unreactive. Alternatively, the terminus may include a reactive group. When the terminus is a reactive group, the reactive group is either selected such that it is unreactive under the conditions of formation of the final oligomer or during covalent attachment of the oligomer to a small molecule drug, or it is protected as necessary. One common end-functional group is hydroxyl or —OH, particularly for oligoethylene oxides.

The water-soluble, non-peptidic oligomer (e.g., “POLY” in various structures provided herein) can have any of a number of different geometries. For example, it can be linear, branched, or forked. Most typically, the water-soluble, non-peptidic oligomer is linear or is branched, for example, having one branch point. Although much of the discussion herein is focused upon poly(ethylene oxide) as an illustrative oligomer, the discussion and structures presented herein can be readily extended to encompass any water-soluble, non-peptidic oligomers described above.

The molecular weight of the water-soluble, non-peptidic oligomer, excluding the linker portion, is generally relatively low. Exemplary values of the molecular weight of the water-soluble polymer include: below about 1500; below about 1450; below about 1400; below about 1350; below about 1300; below about 1250; below about 1200; below about 1150; below about 1100; below about 1050; below about 1000; below about 950; below about 900; below about 850; below about 800; below about 750; below about 700; below about 650; below about 600; below about 550; below about 500; below about 450; below about 400; below about 350; below about 300; below about 250; below about 200; and below about 100 Daltons.

Exemplary ranges of molecular weights of the water-soluble, non-peptidic oligomer (excluding the linker) include: from about 100 to about 1400 Daltons; from about 100 to about 1200 Daltons; from about 100 to about 800 Daltons; from about 100 to about 500 Daltons; from about 100 to about 400 Daltons; from about 200 to about 500 Daltons; from about 200 to about 400 Daltons; from about 75 to 1000 Daltons; and from about 75 to about 750 Daltons.

Preferably, the number of monomers in the water-soluble, non-peptidic oligomer falls within one or more of the following ranges: between about 1 and about 30 (inclusive); between about 1 and about 25; between about 1 and about 20; between about 1 and about 15; between about 1 and about 12; between about 1 and about 10. In certain instances, the number of monomers in series in the oligomer (and the corresponding conjugate) is one of 1, 2, 3, 4, 5, 6, 7, or 8. In additional embodiments, the oligomer (and the corresponding conjugate) contains 9, 10, 11, 12, 13, 14, 15, 16, 17, 18, 19, or 20 monomers. In yet further embodiments, the oligomer (and the corresponding conjugate) possesses 21, 22, 23, 24, 25, 26, 27, 28, 29 or 30 monomers in series. Thus, for example, when the water-soluble and non-peptidic polymer includes CH₃—(OCH₂CH₂)ₙ— or H—(OCH₂CH₂)ₙ—, “n” is an integer that can be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and can fall within one or more of the following ranges: between about 1 and about 25; between about 1 and about 20; between about 1 and about 15; between about 1 and about 12; between about 1 and about 10.

When the water-soluble, non-peptidic oligomer has 1, 2, 3, 4, 5, 6, 7, 8, 9, or 10 monomers, these values correspond to a methoxy end-capped oligo(ethylene oxide) having a molecular weight of about 75, 119, 163, 207, 251, 295, 339, 383, 427, and 471 Daltons, respectively. When the oligomer has 11, 12, 13, 14, or 15 monomers, these values correspond to methoxy end-capped oligo(ethylene oxide) having molecular weights corresponding to about 515, 559, 603, 647, and 691 Daltons, respectively.

When the water-soluble, non-peptidic oligomer is attached to lidocaine or lidocaine derivative (in contrast to the step-wise addition of one or more monomers to effectively “grow” the oligomer onto lidocaine or the derivative thereof), it is preferred that the composition containing an activated form of the water-soluble, non-peptidic oligomer be monodisperse. In those instances where a bimodal composition is employed, the composition will possess a bimodal distribution centering around any two of the above numbers of monomers. For instance, a bimodal oligomer may have any one of the following exemplary combinations of monomer subunits: 1-2, 1-3, 1-4, 1-5, 1-6, 1-7, 1-8, 1-9, 1-10, and so forth; 2-3, 2-4, 2-5, 2-6, 2-7, 2-8, 2-9, 2-10, and so forth; 3-4, 3-5, 3-6, 3-7, 3-8, 3-9, 3-10, and so forth; 4-5, 4-6, 4-7, 4-8, 4-9, 4-10, and so forth; 5-6, 5-7, 5-8, 5-9, 5-10, and so forth; 6-7, 6-8, 6-9, 6-10, and so forth; 7-8, 7-9, 7-10, and so forth; and 8-9, 8-10, and so forth.

In some instances, the composition containing an activated form of the water-soluble, non-peptidic oligomer will be trimodal or even tetramodal, possessing a range of monomers units as previously described. Oligomer compositions possessing a well-defined mixture of oligomers (i.e., being bimodal, trimodal, tetramodal, and so forth) can be prepared by mixing purified monodisperse oligomers to obtain a desired profile of oligomers (a mixture of two oligomers differing only in the number of monomers is bimodal; a mixture of three oligomers differing only in the number of monomers is trimodal; a mixture of four oligomers differing only in the number of monomers is tetramodal), or alternatively, can be obtained from column chromatography of a polydisperse oligomer by recovering the “center cut,” to obtain a mixture of oligomers in a desired and defined molecular weight range.

It is preferred that the water-soluble, non-peptidic oligomer is obtained from a composition that is preferably unimolecular or monodisperse. That is, the oligomers in the composition possess the same discrete molecular weight value rather than a distribution of molecular weights. Some monodisperse oligomers can be purchased from commercial sources such as those available from Sigma-Aldrich, or alternatively, can be prepared directly from commercially available starting materials such as Sigma-Aldrich. Water-soluble and non-peptidic oligomers can be prepared as described in Chen Y., Baker, G. L., J. Org. Chem., 6870-6873 (1999), WO 02/098949, and U.S. Patent Application Publication 2005/0136031.

When present, the spacer moiety (through which the water-soluble and non-peptidic polymer is attached to lidocaine or a lidocaine derivative) may be a single bond, a single atom, such as an oxygen atom or a sulfur atom, two atoms, or a number of atoms. A spacer moiety is typically but is not necessarily linear in nature. The spacer moiety, “X,” is hydrolytically stable, and is preferably also enzymatically stable. Preferably, the spacer moiety “X” is one having a chain length of less than about 12 atoms, and preferably less than about 10 atoms, and even more preferably less than about 8 atoms and even more preferably less than about 5 atoms, whereby length is meant the number of atoms in a single chain, not counting substituents. For instance, a urea linkage such as this, R_{oligomer}—NH—(C═O)—NH—R′_{drug}, is considered to have a chain length of 3 atoms (—NH—C(O)—NH—). In selected embodiments, the linkage does not comprise further spacer groups.

In some instances, the spacer moiety “X” comprises an ether, amide, urethane, amine, thioether, urea, or a carbon-carbon bond. Functional groups such as those discussed below, and illustrated in the examples, are typically used for forming the linkages. The spacer moiety may less preferably also comprise (or be adjacent to or flanked by) other atoms, as described further below.

More specifically, in selected embodiments, a spacer moiety of the invention, X, may be any of the following: “-” (i.e., a covalent bond, that may be stable or degradable, between lidocaine (or the lidocaine derivative) and the water-soluble, non-peptidic oligomer), —C(O)O—, —OC(O)—, —CH₂—C(O)O—, —CH₂—OC(O)—, —C(O)O—CH₂—, —OC(O)—CH₂—, —O—, —NH—, —S—, —C(O)—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, bivalent cycloalkyl group, —N(R⁶)—, R⁶ is H or an organic radical selected from the group consisting of alkyl, substituted alkyl, alkenyl, substituted alkenyl, alkynyl, substituted alkynyl, aryl and substituted aryl.

For purposes of the present invention, however, a group of atoms is not considered a linkage when it is immediately adjacent to an oligomer segment, and the group of atoms is the same as a monomer of the oligomer such that the group would represent a mere extension of the oligomer chain.

The linkage “X” between the water-soluble, non-peptidic oligomer and the small molecule is typically formed by reaction of a functional group on a terminus of the oligomer (or nascent oligomer when it is desired to “grow” the oligomer onto the lidocaine or a derivative thereof) with a corresponding functional group within lidocaine. Illustrative reactions are described briefly below. For example, an amino group on an oligomer may be reacted with a carboxylic acid or an activated carboxylic acid derivative on the small molecule, or vice versa, to produce an amide linkage. Alternatively, reaction of an amine on an oligomer with an activated carbonate (e.g. succinimidyl or benzotriazyl carbonate) on the drug, or vice versa, forms a carbamate linkage. Reaction of an amine on an oligomer with an isocyanate (R—N═C═O) on a drug, or vice versa, forms a urea linkage (R—NH—(C═O)—NH—R′). Further, reaction of an alcohol (alkoxide) group on an oligomer with an alkyl halide, or halide group within a drug, or vice versa, forms an ether linkage. In yet another coupling approach, a small molecule having an aldehyde function is coupled to an oligomer amino group by reductive amination, resulting in formation of a secondary amine linkage between the oligomer and the small molecule.

A particularly preferred water-soluble, non-peptidic oligomer is an oligomer bearing an aldehyde functional group. In this regard, the oligomer will have the following structure: CH₃O—(CH₂—CH₂—O)ₙ—(CH₂)ₚ—C(O)H, wherein (n) is one of 1, 2, 3, 4, 5, 6, 7, 8, 9 and 10 and (p) is one of 1, 2, 3, 4, 5, 6 and 7. Preferred (n) values include 3, 5 and 7 and preferred (p) values 2, 3 and 4. In addition, the carbon atom alpha to the —C(O)H moiety can optionally be substituted with alkyl.

Typically, all but one termini of the water-soluble, non-peptidic oligomer not bearing a functional group is capped to render it unreactive. When the oligomer includes a further functional group at a terminus other than that intended for formation of a conjugate, that group is either selected such that it is unreactive under the conditions of formation of the linkage “X,” or it is protected during the formation of the linkage “X.”

As stated above, the water-soluble, non-peptidic oligomer includes at least one functional group prior to conjugation. The functional group typically comprises an electrophilic or nucleophilic group for covalent attachment to a small molecule, depending upon the reactive group contained within or introduced into the small molecule. Examples of nucleophilic groups that may be present in either the oligomer or the small molecule include hydroxyl, amine, hydrazine (—NHNH₂), hydrazide (—C(O)NHNH₂), and thiol. Preferred nucleophiles include amine, hydrazine, hydrazide, and thiol, particularly amine. Most small molecule drugs for covalent attachment to an oligomer will possess a free hydroxyl, amino, thio, aldehyde, ketone, or carboxyl group.

Examples of electrophilic functional groups that may be present in either the oligomer or the small molecule include carboxylic acid, carboxylic ester, particularly imide esters, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, and halosilane. More specific examples of these groups include succinimidyl ester or carbonate, imidazoyl ester or carbonate, benzotriazole ester or carbonate, vinyl sulfone, chloroethylsulfone, vinylpyridine, pyridyl disulfide, iodoacetamide, glyoxal, dione, mesylate, tosylate, and tresylate (2,2,2-trifluoroethanesulfonate).

Also included are sulfur analogs of several of these groups, such as thione, thione hydrate, thioketal, is 2-thiazolidine thione, etc., as well as hydrates or protected derivatives of any of the above moieties (e.g. aldehyde hydrate, hemiacetal, acetal, ketone hydrate, hemiketal, ketal, thioketal, thioacetal).

An “activated derivative” of a carboxylic acid refers to a carboxylic acid derivative that reacts readily with nucleophiles, generally much more readily than the underivatized carboxylic acid. Activated carboxylic acids include, for example, acid halides (such as acid chlorides), anhydrides, carbonates, and esters. Such esters include imide esters, of the general form —(CO)O—N[(CO)—]₂; for example, N-hydroxysuccinimidyl (NHS) esters or N-hydroxyphthalimidyl esters. Also preferred are imidazolyl esters and benzotriazole esters. Particularly preferred are activated propionic acid or butanoic acid esters, as described in co-owned U.S. Pat. No. 5,672,662. These include groups of the form —(CH₂)₂₋₃C(═O)O-Q, where Q is preferably selected from N-succinimide, N-sulfosuccinimide, N-phthalimide, N-glutarimide, N-tetrahydrophthalimide, N-norbornene-2,3-dicarboximide, benzotriazole, 7-azabenzotriazole, and imidazole.

Other preferred electrophilic groups include succinimidyl carbonate, maleimide, benzotriazole carbonate, glycidyl ether, imidazoyl carbonate, p-nitrophenyl carbonate, acrylate, tresylate, aldehyde, and orthopyridyl disulfide.

These electrophilic groups are subject to reaction with nucleophiles, e.g., hydroxy, thio, or amino groups, to produce various bond types. Preferred for the present invention are reactions which favor formation of a hydrolytically stable linkage. For example, carboxylic acids and activated derivatives thereof, which include orthoesters, succinimidyl esters, imidazolyl esters, and benzotriazole esters, react with the above types of nucleophiles to form esters, thioesters, and amides, respectively, of which amides are the most hydrolytically stable. Carbonates, including succinimidyl, imidazolyl, and benzotriazole carbonates, react with amino groups to form carbamates. Isocyanates (R—N═C═O) react with hydroxyl or amino groups to form, respectively, carbamate (RNH—C(O)—OR′) or urea (RNH—C(O)—NHR′) linkages. Aldehydes, ketones, glyoxals, diones and their hydrates or alcohol adducts (i.e., aldehyde hydrate, hemiacetal, acetal, ketone hydrate, hemiketal, and ketal) are preferably reacted with amines, followed by reduction of the resulting imine, if desired, to provide an amine linkage (reductive amination).

Several of the electrophilic functional groups include electrophilic double bonds to which nucleophilic groups, such as thiols, can be added, to form, for example, thioether bonds. These groups include maleimides, vinyl sulfones, vinyl pyridine, acrylates, methacrylates, and acrylamides. Other groups comprise leaving groups that can be displaced by a nucleophile; these include chloroethyl sulfone, pyridyl disulfides (which include a cleavable S—S bond), iodoacetamide, mesylate, tosylate, thiosulfonate, and tresylate. Epoxides react by ring opening by a nucleophile, to form, for example, an ether or amine bond. Reactions involving complementary reactive groups such as those noted above on the oligomer and the small molecule are utilized to prepare the conjugates of the invention.

In some instances lidocaine or the lidocaine derivative may not have a functional group suited for conjugation. In this instance, it is possible to modify (or “functionalize”) the “original” lidocaine or lidocaine derivative molecule so that it does have a functional group suited for conjugation. For example, lidocaine has an amide group, but an amine group is desired; it is possible to modify the amide group to an amine group by way of a Hofmann rearrangement, Curtius rearrangement (once the amide is converted to an azide) or Lossen rearrangement (once amide is concerted to hydroxamide followed by treatment with tolyene-2-sulfonyl chloride/base).

It is possible to prepare a conjugate of lidocaine or a small molecule derivative thereof bearing a carboxyl group wherein the carboxyl group-bearing small molecule lidocaine or a derivative thereof blocker is coupled to an amino-terminated oligomeric ethylene glycol, to provide a conjugate having an amide group covalently linking lidocaine or the lidocaine derivative to the oligomer. This can be performed, for example, by combining the carboxyl group-bearing small molecule agent with the amino-terminated oligomeric ethylene glycol in the presence of a coupling reagent, (such as dicyclohexylcarbodiimide or “DCC”) in an anhydrous organic solvent.

Further, it is possible to prepare a conjugate of lidocaine and small molecule conjugate of lidocaine deliveries bearing a hydroxyl group wherein the hydroxyl group-bearing lidocaine or derivative thereof is coupled to an oligomeric ethylene glycol halide to result in an ether (—O—) linked small molecule conjugate. This can be performed, for example, by using sodium hydride to deprotonate the hydroxyl group followed by reaction with a halide-terminated oligomeric ethylene glycol.

In another example, it is possible to prepare a conjugate of lidocaine and a small molecule derivative thereof bearing a ketone group by first reducing the ketone group to form the corresponding hydroxyl group. Thereafter, lidocaine and the small molecule derivatives thereof are now bearing a hydroxyl group can be coupled as described herein.

In still another instance, it is possible to prepare a conjugate of lidocaine and small molecule derivatives thereof bearing an amine group. In one approach, the amine group-bearing small molecule compound and an aldehyde-bearing oligomer are dissolved in a suitable buffer after which a suitable reducing agent (e.g., NaCNBH₃) is added. Following reduction, the result is an amine linkage formed between the amine group of the amine group-containing small molecule of interest and the carbonyl carbon of the aldehyde-bearing oligomer.

In another approach for preparing a conjugate of lidocaine and small molecule derivatives thereof bearing an amine group, a carboxylic acid-bearing oligomer and the amine group-bearing small molecule of interest are combined, typically in the presence of a coupling reagent (e.g., DCC). The result is an amide linkage formed between the amine group of the amine group-containing small molecule and the carbonyl of interest the carboxylic acid-bearing oligomer.

Exemplary compounds of the invention include those having the following structure:

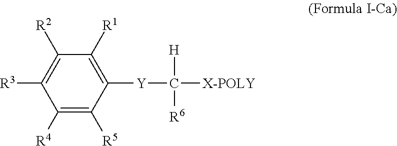

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

Y is selected from the group consisting of —O—, —O—CH₂—, —CH₂—O—, —NH—, —NHC(O)—, —C(O)NH—, —CH₂— and —CH₂—CH₂—;

X is a spacer moiety; and

POLY is a water-soluble, non-peptidic oligomer.

Additional exemplary compounds of the invention include those having the following structure:

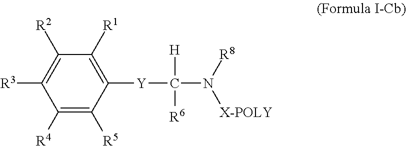

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁸ is hydrogen or lower alkyl;

Y is selected from the group consisting of —O—, —O—CH₂—, —CH₂—O—, —NH—, —NHC(O)—, —C(O)NH—, —CH₂— and —CH₂—CH₂—;

X is a spacer moiety; and

POLY is a water-soluble, non-peptidic oligomer.

Further exemplary compounds of the invention include those having the following structure:

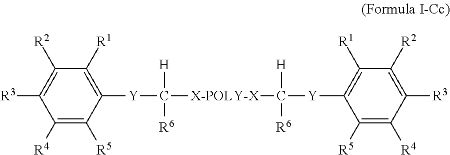

wherein:

each R¹ is hydrogen or lower alkyl (preferably methyl);

each R² is hydrogen or lower alkyl (preferably hydrogen);

each R³ is hydrogen or lower alkyl (preferably hydrogen);

each R⁴ is hydrogen or lower alkyl (preferably hydrogen);

each R⁵ is hydrogen or lower alkyl (preferably methyl);

each R⁶ is hydrogen or lower alkyl (preferably methyl);

each Y is selected from the group consisting of —O—, —O—CH₂—, —CH₂—O—, —NH—, —NHC(O)—, —C(O)NH—, —CH₂— and —CH₂—CH₂—;

each X is a spacer moiety; and

POLY is a water-soluble, non-peptidic oligomer.

Still further exemplary compounds of the invention include those having the following structure:

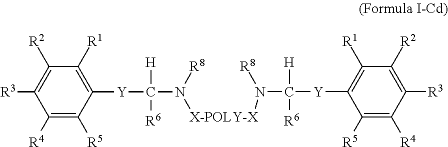

wherein:

each R¹ is hydrogen or lower alkyl (preferably methyl);

each R² is hydrogen or lower alkyl (preferably hydrogen);

each R³ is hydrogen or lower alkyl (preferably hydrogen);

each R⁴ is hydrogen or lower alkyl (preferably hydrogen);

each R⁵ is hydrogen or lower alkyl (preferably methyl);

each R⁶ is hydrogen or lower alkyl (preferably methyl);

each R⁸ is hydrogen or lower alkyl;

each Y is selected from the group consisting of —O—, —O—CH₂—, —CH₂—O—, —NH—, —NHC(O)—, —C(O)NH—, —CH₂— and —CH₂—CH₂—;

each X is a spacer moiety; and

POLY is a water-soluble, non-peptidic oligomer.

Additional exemplary compounds of the invention include those having the following structure:

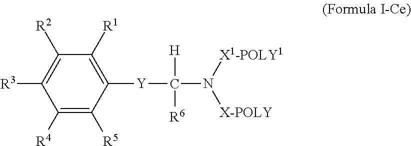

wherein:

R¹ is hydrogen or lower alkyl (preferably methyl);

R² is hydrogen or lower alkyl (preferably hydrogen);

R³ is hydrogen or lower alkyl (preferably hydrogen);

R⁴ is hydrogen or lower alkyl (preferably hydrogen);

R⁵ is hydrogen or lower alkyl (preferably methyl);

R⁶ is hydrogen or lower alkyl (preferably methyl);

R⁸ is hydrogen or lower alkyl;

Y is selected from the group consisting of —O—, —O—CH₂—, —CH₂—O—, —NH—, —NHC(O)—, —C(O)NH—, —CH₂— and —CH₂—CH₂—;

X is a spacer moiety;

X¹ is a spacer moiety;

POLY is a water-soluble, non-peptidic oligomer; and

POLY¹ is a spacer moiety.

The conjugates of the invention can exhibit a reduced blood-brain barrier crossing rate. Moreover, the conjugates maintain at least about 5%, 10%, 20%, 30%, 40%, 50%, 60%, 70%, or more of the bioactivity of the unmodified parent small molecule drug.

While it is believed that the full scope of the conjugates disclosed herein behave as described, an optimally sized oligomer can be identified as follows.

First, an oligomer is conjugated to the small molecule drug. Preferably, the drug is orally bioavailable, and on its own, exhibits a non-negligible blood-brain barrier crossing rate. Next, the ability of the conjugate to cross the blood-brain barrier is determined using an appropriate model and compared to that of the unmodified parent drug. If the results are favorable, that is to say, if, for example, the rate of crossing is significantly reduced, then the bioactivity of conjugate is further evaluated. Preferably, the compounds according to the invention maintain a significant degree of bioactivity relative to the parent drug, i.e., greater than about 30% of the bioactivity of the parent drug, or even more preferably, greater than about 50% of the bioactivity of the parent drug.

The above steps are repeated one or more times using oligomers of the same monomer type but having a different number of subunits and the results compared.

For each conjugate whose ability to cross the blood-brain barrier is reduced in comparison to the non-conjugated small molecule drug, its oral bioavailability is then assessed. Based upon these results, that is to say, based upon the comparison of conjugates of oligomers of varying size to a given small molecule at a given position or location within the small molecule, it is possible to determine the size of the oligomer most effective in providing a conjugate having an optimal balance between reduction in biological membrane crossing, oral bioavailability, and bioactivity. The small size of the oligomers makes such screenings feasible and allows one to effectively tailor the properties of the resulting conjugate. By making small, incremental changes in oligomer size and utilizing an experimental design approach, one can effectively identify a conjugate having a favorable balance of reduction in biological membrane crossing rate, bioactivity, and oral bioavailability. In some instances, attachment of an oligomer as described herein is effective to actually increase oral bioavailability of the drug.

For example, one of ordinary skill in the art, using routine experimentation, can determine a best suited molecular size and linkage for improving oral bioavailability by first preparing a series of oligomers with different weights and functional groups and then obtaining the necessary clearance profiles by administering the conjugates to a patient and taking periodic blood and/or urine sampling. Once a series of clearance profiles have been obtained for each tested conjugate, a suitable conjugate can be identified.

Animal models (rodents and dogs) can also be used to study oral drug transport. In addition, non-in vivo methods include rodent everted gut excised tissue and Caco-2 cell monolayer tissue-culture models. These models are useful in predicting oral drug bioavailability.

To determine whether the lidocaine derivative itself or the conjugate of lidocaine or a derivative thereof has activity (such as analgesic activity), it is possible to test such a compound. For example, the compound of interest can be administered to a mouse topically and analgesia assessed as described in Kolesnikov et al. (1999) J. Pharmacol. Exp. Ther. 290: 247-252. Briefly, the distal portion of the tail (2-3 cm) is immersed in a DMSO solution containing the compound of interest for the stated time, typically two minutes. Testing is performed on the portion of the tail immersed in the treatment solution, because the analgesic actions of agents administered in this manner are restricted to the exposed portions of the tail. Antinociception, or analgesia, is defined as a tail-flick latency for an individual animal that is twice its baseline latency or greater. Baseline latencies typically range from 2.5 to 3.0 seconds, with a maximum cutoff latency of 10 seconds to minimize tissue damage in analgesic animals. ED₅₀ values can be determined.

In another approach for evaluating analgesic activity of the lidocaine derivative itself or the conjugate of lidocaine or a derivative thereof, a “writhing test” can be conducted. Briefly, a 0.7% acetic acid solution is administered (i.p.) to a mouse and the numbers of writhing responses are counted for ten minutes. Thereafter, the compound to be tested is administered [by, for example, injection (e.g., subcutaneous injection)] to the mouse and antinociception is quantified as percent inhibition using the following formula: % inhibition=[(control responses−test responses)/control responses]×100. An exemplary “writhing test” is set forth in the Experimental.

The present invention also includes pharmaceutical preparations comprising a conjugate as provided herein in combination with a pharmaceutical excipient. Generally, the conjugate itself will be in a solid form (e.g., a precipitate), which can be combined with a suitable pharmaceutical excipient that can be in either solid or liquid form.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Pharmaceutically acceptable acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate in the composition will vary depending on a number of factors, but will optimally be a therapeutically effective dose when the composition is stored in a unit dose container. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, excipients will be present in the composition in an amount of about 1% to about 99% by weight, preferably from about 5%-98% by weight, more preferably from about 15-95% by weight of the excipient, with concentrations less than 30% by weight most preferred.

These foregoing pharmaceutical excipients along with other excipients and general teachings regarding pharmaceutical compositions are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The pharmaceutical compositions can take any number of forms and the invention is not limited in this regard. Exemplary preparations are most preferably in a form suitable for oral administration such as a tablet, caplet, capsule, gel cap, troche, dispersion, suspension, solution, elixir, syrup, lozenge, transdermal patch, spray, suppository, and powder.

Oral dosage forms are preferred for those conjugates that are orally active, and include tablets, caplets, capsules, gel caps, suspensions, solutions, elixirs, and syrups, and can also comprise a plurality of granules, beads, powders or pellets that are optionally encapsulated. Such dosage forms are prepared using conventional methods known to those in the field of pharmaceutical formulation and described in the pertinent texts.

Tablets and caplets, for example, can be manufactured using standard tablet processing procedures and equipment. Direct compression and granulation techniques are preferred when preparing tablets or caplets containing the conjugates described herein. In addition to the conjugate, the tablets and caplets will generally contain inactive, pharmaceutically acceptable carrier materials such as binders, lubricants, disintegrants, fillers, stabilizers, surfactants, coloring agents, flow agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage forms, in which case the conjugate-containing composition can be encapsulated in the form of a liquid or gel (e.g., in the case of a gel cap) or solid (including particulates such as granules, beads, powders or pellets). Suitable capsules include hard and soft capsules, and are generally made of gelatin, starch, or a cellulosic material. Two-piece hard gelatin capsules are preferably sealed, such as with gelatin bands or the like.

Included are parenteral formulations in the substantially dry form (typically as a lyophilizate or precipitate, which can be in the form of a powder or cake), as well as formulations prepared for injection, which are typically liquid and requires the step of reconstituting the dry form of parenteral formulation. Examples of suitable diluents for reconstituting solid compositions prior to injection include bacteriostatic water for injection, dextrose 5% in water, phosphate-buffered saline, Ringer's solution, saline, sterile water, deionized water, and combinations thereof.

In some cases, compositions intended for parenteral administration can take the form of nonaqueous solutions, suspensions, or emulsions, each typically being sterile. Examples of nonaqueous solvents or vehicles are propylene glycol, polyethylene glycol, vegetable oils, such as olive oil and corn oil, gelatin, and injectable organic esters such as ethyl oleate.

The parenteral formulations described herein can also contain adjuvants such as preserving, wetting, emulsifying, and dispersing agents. The formulations are rendered sterile by incorporation of a sterilizing agent, filtration through a bacteria-retaining filter, irradiation, or heat.

The conjugate can also be administered through the skin using conventional transdermal patch or other transdermal delivery system, wherein the conjugate is contained within a laminated structure that serves as a drug delivery device to be affixed to the skin. In such a structure, the conjugate is contained in a layer, or “reservoir,” underlying an upper backing layer. The laminated structure can contain a single reservoir, or it can contain multiple reservoirs.

The conjugate can also be formulated into a suppository for rectal administration. With respect to suppositories, the conjugate is mixed with a suppository base material which is (e.g., an excipient that remains solid at room temperature but softens, melts or dissolves at body temperature) such as coca butter (theobroma oil), polyethylene glycols, glycerinated gelatin, fatty acids, and combinations thereof. Suppositories can be prepared by, for example, performing the following steps (not necessarily in the order presented): melting the suppository base material to form a melt; incorporating the conjugate (either before or after melting of the suppository base material); pouring the melt into a mold; cooling the melt (e.g., placing the melt-containing mold in a room temperature environment) to thereby form suppositories; and removing the suppositories from the mold.

The invention also provides a method for administering a conjugate as provided herein to a patient suffering from a condition that is responsive to treatment with the conjugate. The method comprises administering, generally orally, a therapeutically effective amount of the conjugate (preferably provided as part of a pharmaceutical preparation). Other modes of administration are also contemplated, such as pulmonary, nasal, buccal, rectal, sublingual, transdermal, and parenteral. As used herein, the term “parenteral” includes subcutaneous, intravenous, intra-arterial, intraperitoneal, intracardiac, intrathecal, and intramuscular injection, as well as infusion injections.

In instances where parenteral administration is utilized, it may be necessary to employ somewhat bigger oligomers than those described previously, with molecular weights ranging from about 500 to 30K Daltons (e.g., having molecular weights of about 500, 1000, 2000, 2500, 3000, 5000, 7500, 10000, 15000, 20000, 25000, 30000 or even more).

The method of administering may be used to treat any condition that can be remedied or prevented by administration of the particular conjugate. Those of ordinary skill in the art appreciate which conditions a specific conjugate can effectively treat. The actual dose to be administered will vary depend upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 1000 mg, preferably in doses from 0.01 mg/day to 750 mg/day, and more preferably in doses from 0.10 mg/day to 500 mg/day.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

One advantage of administering the conjugates of the present invention is that a reduction in first pass metabolism may be achieved relative to the parent drug. Such a result is advantageous for many orally administered drugs that are substantially metabolized by passage through the gut. In this way, clearance of the conjugate can be modulated by selecting the oligomer molecular size, linkage, and position of covalent attachment providing the desired clearance properties. One of ordinary skill in the art can determine the ideal molecular size of the oligomer based upon the teachings herein. Preferred reductions in first pass metabolism for a conjugate as compared to the corresponding nonconjugated small drug molecule include: at least about 10%, at least about 20%, at least about 30; at least about 40; at least about 50%; at least about 60%, at least about 70%, at least about 80% and at least about 90%.

Thus, the invention provides a method for reducing the metabolism of an active agent. The method comprises the steps of: providing conjugates, each conjugate comprised of a moiety derived from a small molecule drug covalently attached by a stable linkage to a water-soluble oligomer, wherein said conjugate exhibits a reduced rate of metabolism as compared to the rate of metabolism of the small molecule drug not attached to the water-soluble oligomer; and administering the conjugate to a patient. Typically, administration is carried out via one type of administration selected from the group consisting of oral administration, transdermal administration, buccal administration, transmucosal administration, vaginal administration, rectal administration, parenteral administration, and pulmonary administration.

Although useful in reducing many types of metabolism (including both Phase I and Phase II metabolism) can be reduced, the conjugates are particularly useful when the small molecule drug is metabolized by a hepatic enzyme (e.g., one or more of the cytochrome P450 isoforms) and/or by one or more intestinal enzymes.

All articles, books, patents, patent publications and other publications referenced herein are incorporated by reference in their entireties. In the event of an inconsistency between the teachings of this specification and the art incorporated by reference, the meaning of the teachings in this specification shall prevail.

### EXPERIMENTAL

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All chemical reagents referred to in the appended examples are commercially available unless otherwise indicated. The preparation of PEG-mers is described in, for example, U.S. Patent Application Publication No. 2005/0136031.

All ¹H NMR (nuclear magnetic resonance) data was generated by a 300 MHz NMR spectrometer manufactured by Bruker. A list of certain compounds as well as the source of the compounds is provided below.

### Example 1

### PEG-Lidocaine Conjugates

Conjugates of lidocaine can be prepared in accordance with the schematic provided below.

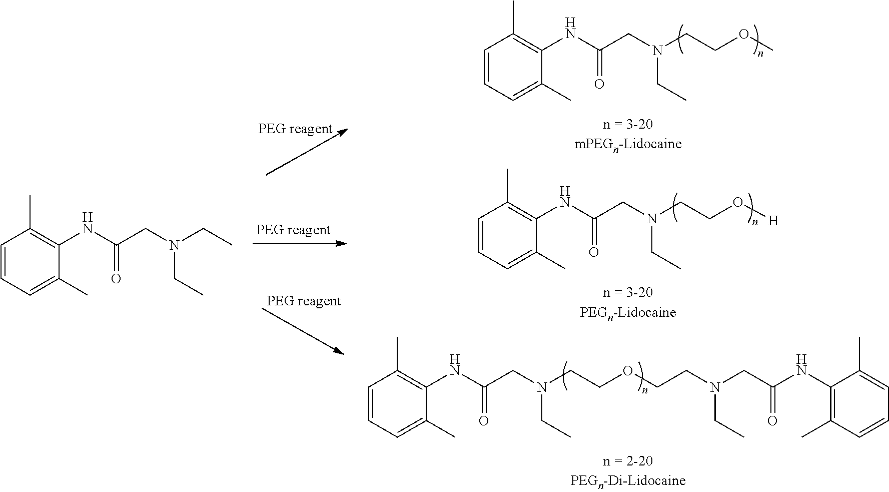

The following materials were used in this Example 1: 2-chloro-2′,6′-acetoxylidide, 99%, Aldrich; ethylamine (“EtNH₂”), 70 wt % solution in water, Aldrich; methanesulfonyl chloride (MsCl), 99.5%, Aldrich; potassium iodide, 99%, Aldrich; potassium carbonate, 99%, Aldrich; PEG₄-di-OH [tetra(ethyleneglycol)], 99%, Aldrich; PEG₅-Di-OH [penta(ethyleneglycol)], 98%, Aldrich; PEG₆-di-OH (Hexaethylene glycol), 95%, Fluka; triethylamine, “TEA,” (99.5%), Aldrich; tetrabutylammonium bromide (“TBAB”), 99%, Aldrich; and trity chloride (TrCl), 98%, Aldrich.

In this Example 1, the following reaction schema were employed.

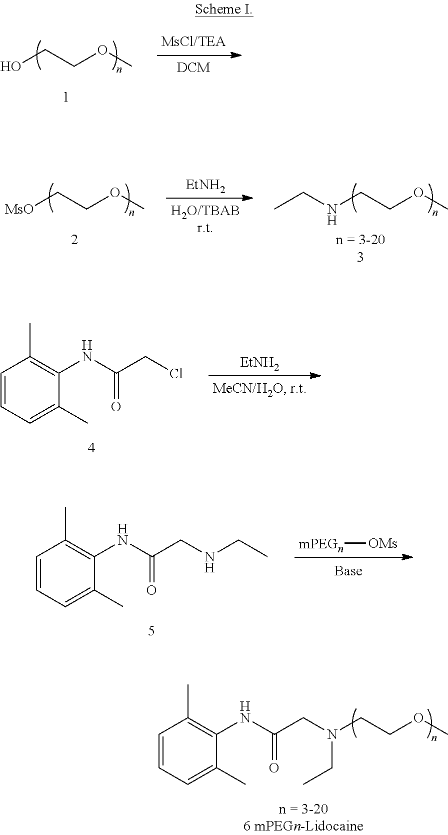

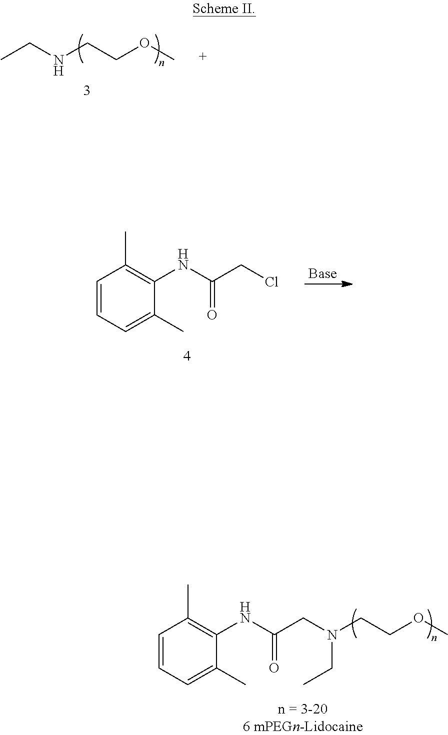

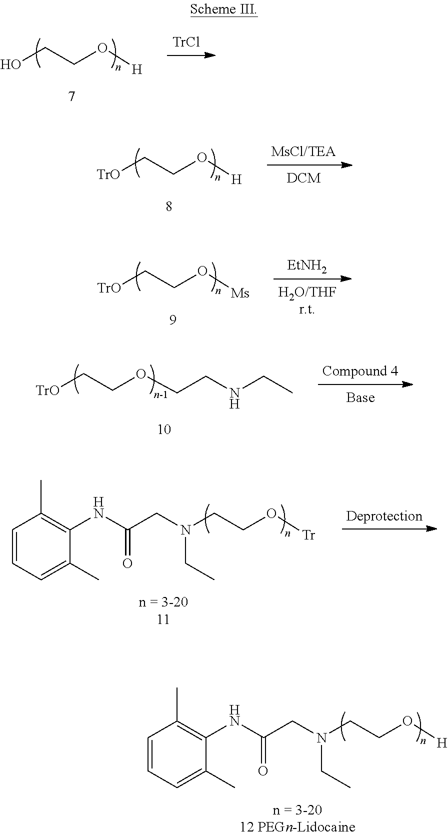

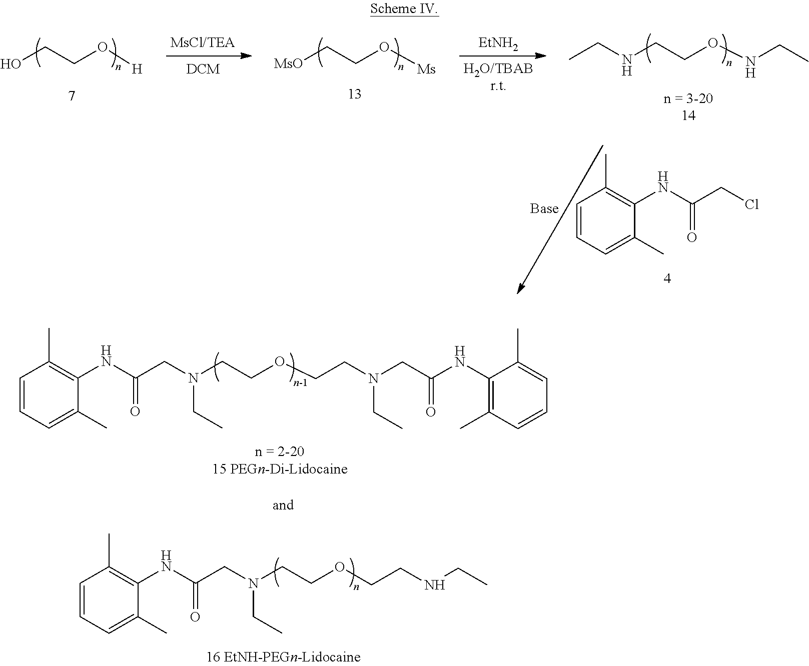

Synthesis of mPEG5-OMs 2 (n=5):

MSCl (2.5 mL, 32 mmol) was added dropwise to a stirring solution of mPEG₅-OH (5.30 g, 21 mmol) and TEA (6 mL, 42.8 mmol) in dichloromethane (“DCM”) (50 mL) at 0° C. After addition, the resulting solution was stirred at room temperature (“r.t.”) for 22 hours. Water (10 mL) was added to quench the reaction and a saturated NaCl solution (˜40 mL) was added. The organic layers were separated and washed with brine (2×45 mL), dried over Na₂SO₄, filtered, and the solvent removed under reduced pressure. The residue was dried under high vacuum to afford the desired product as an oil in quantitative yield. Other mPEGn-OMs (n=3, 4, 6-20) of different sizes were synthesized following the same procedures from the corresponding mPEGn-OH. ¹H-NMR (CDCl₃) 4.38-4.35 (m, 2H), 3.76-3.73 (m, 2H), 3.66-3.60 (m, 14H), 3.55-3.51 (m, 2H), 3.36 (s, 3H), 3.06 (s, 3H).

Synthesis of mPEG3-NHEt 3 (n=3):

Ethylamine (70 wt % solution in water) (10 mL, 123.60 mmol) was added to a stirring solution of mPEG₃-OMs (2.64 g, 10.88 mmol) and K₂CO₃ (7.12 g, 51.0 mmol) in water (10 mL) at 0° C. Tetrabutylammonium bromide (305 mg, 0.94 mmol) was added. The resulting mixture was stirred at room temperature for 67 hours. The mixture was extracted with dichloromethane (3×20 mL). The combined organic layers were washed with brine, dried over anhydrous Na₂SO₄, filtered, and the solvent removed under reduced pressure to afford the desired product in almost quantitative yield. ¹H-NMR (CDCl₃) 3.64-3.51 (m, 10H), 3.36 (s, 3H), 2.76 (t, J=5.1-5.4 Hz, 2H), 2.63 (q, J=7.2H, 2H), 1.08 (t, J=7.2 Hz, 3H).

Synthesis of mPEG4-NHEt 3 (n=4):

Ethylamine (70 wt % solution in water) (8 mL, 98.9 mmol) was added to a stirring solution of mPEG₄-OMs (2.75 g, 9.6 mmol) and K₂CO₃ (6.72 g, 48.16 mmol) in water (10 mL) at 0° C. Tetrabutylammonium bromide (268 mg, 0.82 mmol) was added. The resulting mixture was stirred at room temperature for 67 hours. The mixture was extracted with dichloromethane (3×20 mL). The combined organic layers were washed with brine, dried over anhydrous Na₂SO₄, filtered, and the solvent removed under reduced pressure to afford the desired product in 95% yield. ¹H-NMR (CDCl₃) 3.64-3.51 (m, 14H), 3.36 (s, 3H), 2.76 (t, J=5.1-5.4 Hz, 2H), 2.63 (q, J=7.2H, 2H), 1.09 (t, J=7.2 Hz, 3H).

Synthesis of mPEG5-NHEt 3 (n=5):

Ethylamine (70 wt % solution in water) (5.5 mL, 67.98 mmol) was added to a stirring solution of mPEG₅-OMs (2.14 g, 6.49 mmol) and K₂CO₃ (4.57 g, 32.73 mmol) in water (7 mL) at 0° C. Tetrabutylammonium bromide (121 mg, 0.37 mmol) was added. The resulting mixture was stirred at room temperature for 67 hours. The mixture was extracted with dichloromethane (3×25 mL). The combined organic layers were washed with brine, dried over anhydrous Na₂SO₄, filtered, and the solvent removed under reduced pressure to afford the desired product in 91% yield. ¹H-NMR (CDCl₃) 3.64-3.50 (m, 18H), 3.35 (s, 3H), 2.76 (t, J=5.1-5.4 Hz, 2H), 2.63 (q, J=7.2H, 2H), 1.08 (t, J=7.2 Hz, 3H). Other mPEGn-NHEt (n=6-20) of different sizes were synthesized following the same procedures from the corresponding mPEGn-OMs.

Synthesis of de-ethyl-lidocaine 5:

K₂CO₃ (6.928 g, 49.63 mmol) was dissolved in water (15 mL), cooled to room temperature, and added to a stirring solution of 2-chloro-2′,6′-acetoxylidide 4 (4.559 g, 22.83 mmol) in acetonitrile (70 mL). Then EtNH₂ (70 wt % solution in water, 20 mL, 0.247 mmol) was added. The resulting mixture was stirred at room temperature for 25 hours, concentrated to remove acetonitrile and excess of ethylamine. The remaining solution was mixed with saturated NaCl (20 mL), extracted with dichloromethane (3×25 mL). The combined organic layers were washed with brine, dried over anhydrous Na₂SO₄, filtered, and the solvent removed under reduced pressure to afford the desired product as solid in 98% yield. ¹H-NMR (CDCl₃) 8.81 (br, 1H, NH), 7.07 (s, 3H, Ar—H), 3.44 (s, 3H, CH₂), 2.77 (t, J=7.2 Hz, 2H, CH₂), 2.21 (s, 6H, 2CH₃), 1.16 (t, J=7.2 Hz, 3H, CH₃). LC-MS m/z 206.9 (MH⁺).

Synthesis of mPEG3-lidocaine 6 (n=3):

Crude mPEG3-NHEt 3 (n=3) (464 mg, ˜2.18 mmol) and 2-chloro-2′,6′-acetoxylidide 4 (436 mg, 2.18 mmol) were dissolved in acetonitrile (20 mL) at room temperature. K₂CO₃ (922 mg, 6.6 mmol) and KI (363 mg, 2.16 mmol) were added. The resulting mixture was stirred at room temperature for 48.5 hours, filtered and the solid was washed with MeCN and dichloromethane. The combined solutions were concentrated. The residue was purified with flash column chromatography on silica gel to afford the product (511 mg) in 67% yield. ¹H-NMR (CDCl₃) 9.07 (br, 1H, NH), 7.05 (s, 3H, Ar—H), 3.60-3.56 (m, 4H, 2 CH₂), 3.47-3.44 (m, 2H, CH₂), 3.36 (s, 4H, 2 CH₂), 3.30 (s, 5H, CH₃ and CH₂), 2.83 (t, J=5.4 Hz, 2H, CH₂), 2.71 (q, 2H, J=7.2 Hz, CH₂), 2.22 (s, 6H, 2CH₃), 1.11 (t, J=7.2 Hz, 3H, CH₃). LC-MS m/z 353.2 (MH⁺).

Synthesis of mPEG4-lidocaine 6 (n=4):

Crude mPEG4-NHEt 3 (n=4) (624 mg, ˜2.4 mmol) and 2-chloro-2′,6′-acetoxylidide 4 (425 mg, 2.13 mmol) were dissolved in acetonitrile (10 mL) at room temperature. A solution of K₂CO₃ (812 mg, 5.82 mmol) and KI (428 mg, 2.55 mmol) in water (15 mL) was added. The resulting mixture was stirred at 60° C. for 22.5 hours, cooled and concentrated to remove the organic solvents. The remaining solution was mixed with a saturated NaCl solution (10 mL), extracted with dichloromethane (3×20 mL). The combined solutions were concentrated. The residue was purified with flash column chromatography on silica gel to afford the product (497 mg) in 59% yield. ¹H-NMR (CDCl₃) 9.07 (br, 1H, NH), 7.05 (s, 3H, Ar—H), 3.60-3.35 (m, 14H, 7 CH₂), 3.30 (s, 3H, CH₃), 3.11 (s, 2H, CH₂), 2.83 (t, J=5.4 Hz, 2H, CH₂), 2.71 (q, 2H, J=7.2 Hz, CH₂), 2.22 (s, 6H, 2CH₃), 1.11 (t, J=7.2 Hz, 3H, CH₃). LC-MS m/z 397.2 (MH⁺).

Synthesis of mPEG5-lidocaine 6 (n=5):

Crude mPEG5-NHEt 3 (n=5) (636.5 mg, ˜2.12 mmol) and 2-chloro-2′,6′-acetoxylidide 4 (386 mg, 1.93 mmol) were dissolved in acetonitrile (10 mL) at room temperature. A solution of K₂CO₃ (832 mg, 5.96 mmol) and KI (390 mg, 2.33 mmol) in water (15 mL) was added. The resulting mixture was stirred at 60° C. for 18 hours, cooled and concentrated to remove the organic solvents. The remaining solution was mixed with a saturated NaCl solution (10 mL), extracted with dichloromethane (3×20 mL). The combined solutions were concentrated. The residue was purified with flash column chromatography on silica gel to afford the product (358 mg) in 42% yield. ¹H-NMR (CDCl₃) 9.07 (br, 1H, NH), 7.05 (s, 3H, Ar—H), 3.63-3.36 (m, 18H, 9 CH₂), 3.36 (s, 3H, CH₃), 3.30 (s, 2H, CH₂), 2.83 (t, J=5.4 Hz, 2H, CH₂), 2.71 (q, 2H, J=7.2 Hz, CH₂), 2.21 (s, 6H, 2CH₃), 1.11 (t, J=7.2 Hz, 3H, CH₃). LC-MS m/z 441.1 (MH⁺).

Synthesis of mPEG6-lidocaine 6 (n=6):

Crude mPEG6-NHEt 3 (n=6) (908 mg, 2.61 mmol) and 2-chloro-2′,6′-acetoxylidide 4 (458 mg, 2.29 mmol) were dissolved in acetonitrile (15 mL) at room temperature. And then a solution K₂CO₃ (802 mg, 5.74 mmol) and KI (433 mg, 2.58 mmol) in water (15 mL) was added. The resulting mixture was stirred at 60° C. for 22 hours, cooled and concentrated to remove the organic solvents. The remaining solution was mixed with saturated NaCl solution (10 mL), extracted with dichloromethane (3×20 mL). The combined solution was concentrated. The residue was purified with flash column chromatography on silica gel to afford the product (338 mg) in 30% yield. ¹H-NMR (CDCl₃) 9.07 (br, 1H, NH), 7.05 (s, 3H, Ar—H), 3.63-3.43 (m, 20H, 10 CH₂), 3.40-3.36 (m, 2H, CH₂), 3.36 (s, 3H, CH₃), 3.30 (s, 2H, CH₂), 2.83 (t, J=5.4 Hz, 2H, CH₂), 2.71 (q, 2H, J=7.2 Hz, CH₂), 2.21 (s, 6H, 2CH₃), 1.11 (t, J=7.2 Hz, 3H, CH₃). LC-MS m/z 485.1 (MH⁺).

Synthesis of mPEG7-lidocaine 6 (n=7):

A reaction mixture of mPEG7-OMs 2 (n=7) (1.211 g) and de-ethyl-lidocaine 5 (485.6 mg, 2.35 mmol) in the presence of potassium carbonate (453 mg, 3.24 mmol) in water (10 mL) was placed in single-mode focused microwave reactor (CEM) and operated at 120° C. for 30 minutes. The reaction solution was cooled and extracted with dichloromethane (3×15 mL). The combined solution was concentrated. The residue was purified with flash column chromatography on silica gel to afford the product (297 mg) in 24% yield. ¹H-NMR (CDCl₃) 9.07 (br, 1H, NH), 7.05 (s, 3H, Ar—H), 3.63-3.43 (m, 24H, 12 CH₂), 3.39-3.37 (m, 2H, CH₂), 3.37 (s, 3H, CH₃), 3.30 (s, 2H, CH₂), 2.82 (t, J=5.4 Hz, 2H, CH₂), 2.71 (q, 2H, J=7.2 Hz, CH₂), 2.21 (s, 6H, 2CH₃), 1.11 (t, J=7.2 Hz, 3H, CH₃). LC-MS m/z 529.2 (MH⁺).

Synthesis of mPEG8-lidocaine 6 (n=8):

A mixture of crude mPEG8-NHEt 3 (n=8) (995 mg, ˜2.2 mmol), 2-chloro-2′,6′-acetoxylidide 4 (460 mg, 2.3 mmol), K₂CO₃ (1.055 g, 7.56 mmol) and KI (464 mg, 2.77 mmol) in acetonitrile (20 mL) was heated at 85° C. for 1.5 hours. More of mPEG8-NHEt 3 (124 mg, ˜0.27 mmol) in MeCN (0.8 mL) was added. The mixture was heated at 85° C. for another 16.5 hours. The reaction mixture was cooled to room temperature, filtered and washed the solid with MeCN and dichloromethane. The organic solution was collected and concentrated. The residue was purified with flash column chromatography on silica gel to afford the product (1.177 g) in 89% yield. ¹H-NMR (CDCl₃) 9.07 (br, 1H, NH), 7.06 (s, 3H, Ar—H), 3.64-3.44 (m, 28H, 14 CH₂), 3.40-3.37 (m, 2H, CH₂), 3.37 (s, 3H, CH₃), 3.30 (s, 2H, CH₂), 2.83 (t, J=5.4 Hz, 2H, CH₂), 2.71 (q, 2H, J=7.2 Hz, CH₂), 2.22 (s, 6H, 2CH₃), 1.12 (t, J=7.2 Hz, 3H, CH₃). LC-MS m/z 573.1 (MH⁺).

Synthesis of mPEG9-lidocaine 6 (n=9):

A mixture of crude mPEG9-NHEt 3 (n=9) (770 mg, ˜1.6 mmol), 2-chloro-2′,6′-acetoxylidide 4 (303 mg, 1.52 mmol), K₂CO₃ (672 mg, 4.81 mmol) and KI (313 mg, 1.87 mmol) in acetonitrile (15 mL) was heated at 85° C. for 22 hours. The reaction mixture was cooled to room temperature, filtered to remove the solid and washed the solid with MeCN and dichloromethane. The organic solution was collected and concentrated. The residue was purified with flash column chromatography on silica gel to afford the product (820 mg) in 87% yield. ¹H-NMR (CDCl₃) 9.07 (br, 1H, NH), 7.05 (s, 3H, Ar—H), 3.64-3.44 (m, 32H, 16 CH₂), 3.40-3.36 (m, 2H, CH₂), 3.36 (s, 3H, CH₃), 3.30 (s, 2H, CH₂), 2.83 (t, J=5.1 Hz, 2H, CH₂), 2.71 (q, 2H, J=7.2 Hz, CH₂), 2.22 (s, 6H, 2CH₃), 1.12 (t, J=7.2 Hz, 3H, CH₃). LC-MS m/z 617.6 (MH⁺).

Synthesis of TrO-PEG6-OH 8 (n=6):

PEG6-di-OH 7 (n=6) (17.3238 g, 59.52 mmol) was dissolved in toluene (100 mL), concentrated to remove some of toluene (˜80 mL). Anhydrous DMF (125 mL) was added, followed by an addition of DMAP (7.4825 g, 60.02 mmol) and TrCl (13.7833 g, 48.45 mmol). The resulting mixture was heated at 85° C. for 22 hours. The reaction was concentrated to remove the solvents (high vacuum, 50° C.). The residue was mixed with EtOAc (400 mL), washed with 10% K₂CO₃ solution (100 mL), water (100 mL) and brine (300 mL), dried over Na₂CO₃, concentrated. The residue was purified with flash column chromatography on silica gel to result in 14.1 g of product in 55% yield with some side product (TrO-PEG₆-OTr). ¹H-NMR (CDCl₃) 7.46-7.18 (m, 15H, Ar—H), 3.72-3.56 (m, 22H, 11 CH₂), 3.21 (t, J=5.1-5.4 Hz, 2H, CH₂), 2.66 (t, 2H, J=6.0-6.3 Hz, OH). Other TrO-PEGn-OH of different sizes can be synthesized following the same procedures from the corresponding PEGn-di-OH.

Synthesis of TrO-PEG6-OMs 9 (n=6):

Triethylamine (1.7 mL, 12.14 mmol) was added to a solution of TrO-PEG6-OH 8 (n=6) (4.2159 g, 8.04 mmol) in dichloromethane (30 mL), cooled to 0° C. Methanesulfonyl chloride (0.75 mL, 9.6 mmol) was added dropwise. After the addition, the resulting solution was stirred at room temperature for one day. Water was added to quench the reaction. The organic phase was separated and washed with brine (3×100 mL), dried over Na₂SO₄ and concentrated to afford the product as oil (4.399 g) in 91% yield. ¹H-NMR (CDCl₃) 7.46-7.18 (m, 15H, Ar—H), 4.36-4.33 (m, 2H, CH₂), 3.74-3.72 (m, 2H, CH₂), 3.68-3.60 (m, 18H, 9 CH₂), 3.21 (t, J=5.1-5.4 Hz, 2H, CH₂), 3.04 (s, 3H, CH₃). Other TrO-PEGn-OMs of different sizes can be synthesized following the same procedures from the corresponding TrO-PEGn-OH.

Synthesis of TrO-PEG5-NHEt 10 (n=5):

A mixture of TrO-PEG5-OMs (640 mg, 1.15 mmol), EtNH₂ (70 wt % solution in water, 1 mL, 12.36 mmol) in the presence of potassium carbonate (438 mg, 3.14 mmol) in water/THF (2/2 mL) was stirred at room temperature for 66 hours. The mixture was concentrated to remove the organic solvent and excess of methylamine. The aqueous solution was mixed with saturated NaCl solution (˜20 mL), extracted with dichloromethane (3×20 mL). The residue was purified with flash column chromatography on silica gel to afford the product (274 mg) in 47% yield. ¹H-NMR (CDCl₃) 7.43-7.17 (m, 15H, Ar—H), 3.66-3.53 (m, 16H, 8 CH₂), 3.21 (t, J=5.1-5.4 Hz, 2H, CH₂), 2.75 (t, 2H, J=5.1-5.4 Hz, CH₂), 2.61 (q, J=6.9-7.2 Hz, 2H, CH₂), 1.08 (t, 2H, J=6.9-7.2 Hz, CH₂). Other TrO-PEGn-NHEt of different sizes can be synthesized following the same procedures from the corresponding TrO-PEGn-OMs.

Synthesis of TrO-PEG₆-NHEt 10 (n=6):

A mixture of TrO-PEG₆-OMs (2.89 g, 4.79 mmol), EtNH₂ (70 wt % solution in water, 4 mL, 49.44 mmol) in the presence of potassium carbonate (1.706 g, 12.22 mmol) in water/THF (5/5 mL) was stirred at room temperature for 48 h. The mixture was concentrated to remove the organic solvent and excess of methylamine. The aqueous solution was mixed with saturated NaCl solution (˜40 mL), extracted with dichloromethane (3×40 mL). The residue was purified with flash column chromatography on silica gel to afford the product in quantitative yield. ¹H-NMR (CDCl₃) 7.45-7.17 (m, 15H, Ar—H), 3.67-3.55 (m, 20H, 10 CH₂), 3.21 (t, J=5.1 Hz, 2H, CH₂), 2.77 (t, 2H, J=5.1-5.4 Hz, CH₂), 2.63 (q, J=7.2 Hz, 2H, CH₂), 1.08 (t, 2H, J=7.2 Hz, CH₂).

Synthesis of TrO-PEG₅-Lidocaine 11 (n=5):

A mixture of TrO-PEG₅-NHEt 10 (n=5) (274 mg, 0.54 mmol), 2-chloro-2′,6′-acetoxylidide 4 (108 mg, 0.54 mmol), K₂CO₃ (180 mg, 1.29 mmol) and KI (124 mg, 0.74 mmol) in acetonitrile (10 mL) was heated at 85° C. for 4.5 h. The reaction mixture was concentrated to remove the solvent under reduced pressure. The residue was mixed with a mixture solvent of water (10 mL) and dichloromethane (20 mL). The organic phase was separated and the aqueous phase was extracted with dichloromethane (2×20 mL). The combined organic solution was washed with brine, dried over Na₂SO₄, and concentrated. The residue was purified with flash column chromatography on silica gel by using 0-10% MeOH/DCM to afford the product (324 mg) in 90% yield. ¹H-NMR (CDCl₃) 9.07 (br, 1H, NH), 7.45-7.18 (m, 15H, Tr), 7.04 (s, 3H, Ar—H), 3.65-3.37 (m, 16H, 8 CH₂), 3.29 (s, 2H, CH₂), 3.21 (t, J=5.4 Hz, 2H, CH₂), 2.81 (t, J=5.4 Hz, 2H, CH₂), 2.70 (q, 2H, J=7.2 Hz, CH₂), 2.21 (s, 6H, 2CH₃), 1.10 (t, J=7.2 Hz, 3H, CH₃).

Synthesis of TrO-PEG₆-Lidocaine 11 (n=6):

A mixture of TrO-PEG₆-NHEt 10 (n=6) (605 mg, 1.1 mmol), 2-chloro-2′,6′-acetoxylidide 4 (221 mg, 1.1 mmol), K₂CO₃ (318 mg, 2.28 mmol) and KI (225 mg, 1.34 mmol) in acetonitrile (10 mL) was heated at 85° C. for 22.5 h. The reaction mixture was cooled to room temperature, filtered to remove the solid. The solid was washed with DCM and acetonitrile. The combined organic solution was concentrated to remove the solvent under reduced pressure. The residue was dissolved in dichloromethane (40 mL), washed with aq. NaCl solution. The organic solution was concentrated to afford a residue. The residue was purified with flash column chromatography on silica gel by using 0-10% MeOH/DCM to afford the product (750 mg) in 96% yield. ¹H-NMR (CDCl₃) 9.07 (br, 1H, NH), 7.46-7.18 (m, 15H, Tr), 7.05 (s, 3H, Ar—H), 3.65-3.35 (m, 20H, 10 CH₂), 3.30 (s, 2H, CH₂), 3.21 (t, J=5.4 Hz, 2H, CH₂), 2.82 (t, J=5.4 Hz, 2H, CH₂), 2.70 (q, 2H, J=7.2 Hz, CH₂), 2.21 (s, 6H, 2CH₃), 1.10 (t, J=7.2 Hz, 3H, CH₃). LC-MS m/z 713.5 (MH⁺).

Synthesis of HO-PEG5-Lidocaine 12 (n=5):

TrO-PEG5-lidocaine 11 (n=5) (323 mg, 0.48 mmol) was dissolved in 2 N HCl in methanol (20 mL). The mixture was stirred at room temperature for 21 hours. More of 2 N HCl in methanol (20 mL) was added. The mixture was stirred at room temperature for another 19 hours. The mixture was concentrated under reduced pressure to remove organic solvent. The remaining aqueous solution was washed with dichloromethane (10 mL), adjusted to PH 7-8 with saturated potassium carbonate, and then extracted with dichloromethane (3×20 mL). The extraction was washed with brine, dried over Na₂SO₄, and concentrated. The residue was purified with flash column chromatography on silica gel by using 0-10% MeOH/DCM to afford the product (165 mg) in 80% yield. ¹H-NMR (CDCl₃) 9.07 (br, 1H, NH), 7.05 (s, 3H, Ar—H), 3.70-3.38 (m, 18H, 9 CH₂), 3.30 (s, 2H, CH₂), 2.83 (t, J=5.4 Hz, 2H, CH₂), 2.71 (t, J=7.2 Hz, 2H, CH₂), 2.22 (s, 6H, 2CH₃), 1.10 (t, J=7.2 Hz, 3H, CH₃). LC-MS m/z 427.4 (MW).

Synthesis of HO-PEG6-Lidocaine 12 (n=6):

TrO-PEG₆-lidocaine 11 (n=5) (750 mg, 1.05 mmol) was dissolved in 2 N HCl in methanol (30 mL). The mixture was stirred at room temperature for 21 hours. More of 2 N HCl in methanol (20 mL) was added. The mixture was stirred at room temperature for another 19.5 hours. The mixture was concentrated under reduced pressure to remove organic solvent. The remaining aqueous solution was washed with dichloromethane (10 mL), adjusted to PH 7-8 with saturated potassium carbonate, and then extracted with dichloromethane (3×20 mL). The extraction was washed with brine, dried over Na₂SO₄, and concentrated. The residue was purified with flash column chromatography on silica gel by using 0-10% MeOH/DCM to afford the product (396 mg) in 80% yield. ¹H-NMR (CDCl₃) 9.08 (br, 1H, NH), 7.05 (s, 3H, Ar—H), 3.69-3.37 (m, 22H, 11 CH₂), 3.30 (s, 2H, CH₂), 2.83 (t, J=5.4 Hz, 2H, CH₂), 2.71 (t, J=7.2 Hz, 2H, CH₂), 2.21 (s, 6H, 2CH₃), 1.10 (t, J=7.2 Hz, 3H, CH₃). LC-MS m/z 471.4 (MH⁺).

Synthesis of PEG5-di-OMs 13 (n=5):

MSCl (10.5 mL, 134.44 mmol) was added dropwise to a stirred solution of PEG5-di-OH (12.8 g, 52.64 mmol) and triethylamine (22 mL, 157.02 mmol) in dichloromethane (100 mL) at 0° C. After addition, the resulting solution was stirred at room temperature for 22 hours. Water (10 mL) was added to quench the reaction and some saturated NaCl solution (˜30 mL) was added. The organic phase was separated and washed with brine (3×100 mL), dried over Na₂SO₄, and concentrated. The residue was dried under high vacuum to afford the product as an oil in quantitative yield. ¹H-NMR (CDCl₃) 4.38-4.35 (m, 4H, 2 CH₂), 3.76-3.73 (m, 4H, 2 CH₂), 3.67-3.62 (m, 12H, 6 CH₂), 3.12 (s, 6H, 2 CH₃).

Synthesis of PEG6-di-OMs 13 (n=6):

MSCl (5.7 mL, 72.98 mmol) was added dropwise to a stirred solution of PEG6-di-OH (7.89 g, 26.55 mmol) and triethylamine (11.5 mL, 82.10 mmol) in dichloromethane (50 mL) at 0° C. After addition, the resulting solution was stirred at room temperature for 17 hours. Water (10 mL) was added to quench the reaction and some saturated NaCl solution (˜15 mL) was added. The organic phase was separated and washed with brine (2×40 mL), dried over Na₂SO₄, and concentrated. The residue was dried under high vacuum to afford the product as a oil in quantitative yield. ¹H-NMR (CDCl₃) 4.37-4.34 (m, 4H, 2 CH₂), 3.75-3.72 (m, 4H, 2 CH₂), 3.65-3.60 (m, 16H, 8 CH₂), 3.06 (s, 6H, 2 CH₃). Following this procedure, other PEGn-di-OMs was synthesized following the same procedures from the corresponding PEGn-di-OH.

Synthesis of EtNH-PEG5-NHEt 14 (n=5):

Ethylamine (70 wt % solution in water) (25 mL, 0.309 mol) was added to a stirred of PEG5-di-OMs (7.27 g, 18.43 mmol), tetrabutylammonium bromide (380 mg, 1.17 mmol) and K₂CO₃ (10.572 g, 75.73 mmol) in water (15 mL) at 0° C. The resulting mixture was stirred at room temperature for 29 hours. The mixture was diluted with saturated NaCl solution, and then extracted with dichloromethane (3×30 mL). The combined organic solution was washed with brine, dried over anhydrous Na₂SO₄, concentrated to afford the product (5.2934 g) in 98% yield. Other EtNH-PEGn-NHEt of different sizes can be synthesized following the same procedures from the corresponding PEGn-di-OMs. ¹H-NMR (CDCl₃) 3.63-3.55 (m, 16H, 7 CH₂ and 2 NH), 2.79 (t, J=5.1-5.4 Hz, 4H, 2 CH₂), 2.65 (q, 4H, J=7.2 Hz, 2 CH₂), 1.08 (t, 6H, J=7.2 Hz, 2 CH₃).

Synthesis of EtNH-PEG6-NHEt 14 (n=6):

Ethylamine (70 wt % solution in water) (22 mL, 0.272 mol) was added to a stirred of PEG6-di-OMs (8.47 g, 19.32 mmol), tetrabutylammonium bromide (380 mg, 1.17 mmol) and K₂CO₃ (10.6 g, 75.93 mmol) in water (20 mL) at 0° C. The resulting mixture was stirred at 0° C. for 30 minutes, at room temperature for 24 hours. The mixture was diluted with saturated NaCl solution, and then extracted with EtOAc (3×50 mL). The organic solution was washed with brine, dried over Na₂SO₄, concentrated to offer about 3 g of product. The aqueous solution was extracted with dichloromethane (3×40 mL). The combined dichloromethane solution was washed with brine, dried over anhydrous Na₂SO₄, concentrated to afford product. The total of product was 5.429 g, and the yield was 84%. ¹H-NMR (CDCl₃) 3.63-3.55 (m, 20H, 9 CH₂ and 2 NH), 2.77 (t, J=5.1 Hz, 4H, 2 CH₂), 2.63 (q, 4H, J=7.2 Hz, 2 CH₂), 1.08 (t, 6H, J=7.2 Hz, 2 CH₃).

Synthesis of PEG₅-di-lidocaine 15 (n=5):

A mixture of EtNH-PEG5-NHEt 14 (n=5) (578 mg, 1.98 mmol), 2-chloro-2′,6′-acetoxylidide 4 (831 mg, 4.16 mmol), K₂CO₃ (1.73 g, 12.39 mmol) and KI (823 mg, 4.91 mmol) in acetonitrile (30 mL) was heated at 85° C. for 23 h. The reaction mixture was cooled to room temperature, filtered to remove the solid and washed the solid with MeCN and dichloromethane. The organic solution was collected and concentrated. The residue was purified with flash column chromatography on silica gel to afford the product (737 mg) in 61% yield. ¹H-NMR (CDCl₃) 9.06 (br, 2H, 2 NH), 7.05 (s, 3H, Ar—H), 3.58-3.51 (m, 8H, 4 CH₂), 3.40-3.37 (m, 4H, 2 CH₂), 3.30 (s, 4H, 2 CH₂), 3.23 (s, 2H, 2 CH₂), 2.81 (t, J=5.1-5.4 Hz, 4H, 2 CH₂), 2.70 (q, 4H, J=7.2 Hz, 2 CH₂), 2.21 (s, 6H, 2CH₃), 1.11 (t, J=7.2 Hz, 6H, 2 CH₃). LC-MS m/z 615.5 (MH⁺).

Synthesis of PEG6-di-lidocaine 15 (n=6):

A mixture of EtNH-PEG6-NHEt 14 (n=6) (1.00 g, 2.97 mmol), 2-chloro-2′,6′-acetoxylidide 4 (1.197 g, 6.0 mmol), K₂CO₃ (1.70 g, 12.18 mmol) and KI (1.212 g, 7.23 mmol) in acetonitrile (50 mL) was heated at 85° C. for 22.5 hours. The reaction mixture was cooled to room temperature, filtered to remove the solid and washed the solid with MeCN and dichloromethane. The organic solution was collected and concentrated. The residue was purified with flash column chromatography on silica gel to afford the product (973 mg) in 50% yield. ¹H-NMR (CDCl₃) 9.06 (br, 2H, 2 NH), 7.05 (s, 3H, Ar—H), 3.59-3.54 (m, 8H, 4 CH₂), 3.45-3.33 (m, 12H, 6 CH₂), 3.30 (s, 4H, 2 CH₂), 2.82 (t, J=5.4 Hz, 4H, 2 CH₂), 2.71 (q, 4H, J=7.2 Hz, 2 CH₂), 2.21 (s, 6H, 2CH₃), 1.11 (t, J=7.2 Hz, 6H, 2 CH₃). LC-MS m/z 659.5 (MH⁺).

Synthesis of EtNH-PEG5-lidocaine 16 (n=5):

A mixture of EtNH-PEG5-NHEt 14 (n=5) (1.064 g, 3.64 mmol), 2-chloro-2′,6′-acetoxylidide 4 (722 mg, 3.62 mmol), K₂CO₃ (1.06 g, 7.61 mmol) and KI (784 mg, 4.67 mmol) in acetonitrile (40 mL) was heated at 85° C. for 6.5 h. The reaction mixture was cooled to room temperature, filtered to remove the solid and washed the solid with MeCN and dichloromethane. The organic solution was collected and concentrated. The residue was purified with flash column chromatography on silica gel using 0-10% MeOH/DCM to afford the product 16 (n=5) (245 mg) in 15% yield and PEG₅-di-lidocaine 15 (n=5) (664 mg) in 60% yield. Compound 16: ¹H-NMR (CDCl₃) 9.06 (br, 1H, NH), 7.05 (s, 3H, Ar—H), 3.61-3.55 (m, 10H, 5 CH₂), 3.49-3.45 (m, 4H, 2 CH₂), 3.41-3.38 (m, 2H, CH₂), 3.31 (s, 2H, CH₂), 2.85-2.80 (m, 4H, 2 CH₂), 2.75-2.64 (m, 4H, 2 CH₂), 2.21 (s, 6H, 2CH₃), 2.1 (br, 1H, NH), 1.15-1.11 (m, 6H, 2 CH₃). LC-MS m/z 454.4 (MH⁺).

The pKa and Log P for various conjugates prepared in this example were determined and are reported in Table 1.

TABLE 1
pKa and LogP for Various Lidocaine Conjugates
(Conjugate Structure Corresponds to Compound
“6” in Scheme 1 and Scheme II)

| Value of n in | | | |
|---|---|---|---|
| PEGₙ | Sample | pKa | LogP |
| 0 | Lidocaine | 7.95 | 2.39 |
| | | 7.86 (literature) | |
| 3 | mPEG3-Lidocaine | 6.46 | 1.55 |
| 4 | mPEG4-Lidocaine | 6.46 | 1.42 |
| 5 | mPEG5-Lidocaine | 6.46 | 1.01 |
| 6 | mPEG6-Lidocaine | 6.46 | 1.23 |
| 7 | mPEG7-Lidocaine | 6.81 | 1.37 |
| 8 | mPEG8-Lidocaine | 6.43 | 0.63 |
| 9 | mPEG9-Lidocaine | 6.57 | 0.82 |

### Example 2

### PEG-Mexiletine Conjugates

The following materials were used in this Example 1: Mexiletine hydrochloride (97%), Sigma, Lot No. 105K1207; sodium triacetoxyborohydride (95%), Aldrich, Batch No. 07920LD; N, N-diisopropylethylamine, Aldrich, Batch No. 06448PC; cesium carbonate (99%), Aldrich, Lot No. 01527LD; PEG4-di-OH [tetra(ethyleneglycol)] (99%), Aldrich, Batch No. 06322HD; triethylamine (99.5%), Aldrich, Batch No. 09006MD; methanesulfonyl chloride (99.5%), Aldrich, Batch No. 13209KC.

In this Example 2, the following reaction schema were employed.

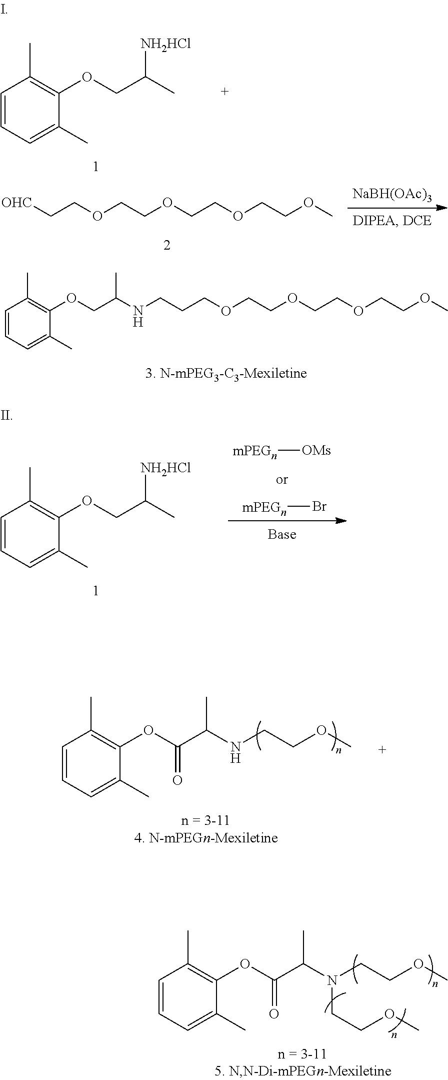

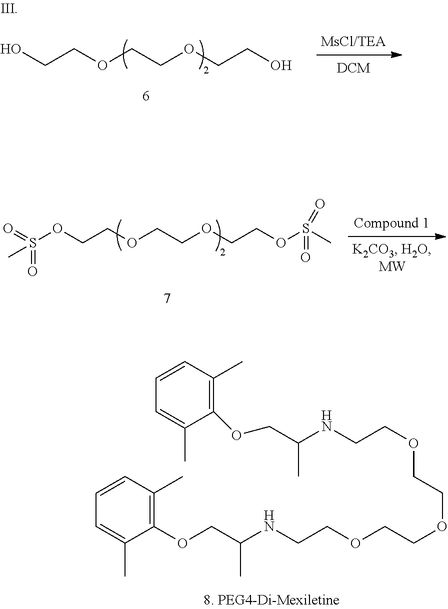

Synthesis of N-mPEG3-C₃-mexiletine 3:

N,N-diisopropylethylamine (0.25 mL, 1.42 mmol) was added to a stirred mixture of mexiletine hydrochloride 1 (309 mg, 1.43 mmol) in 1,2-dichloroethane (5 mL) at room temperature. The mPEG3-propionaldehyde (461 mg, 2.09 mmol) was added and after five minutes, sodium triacetoxyborohydride (624 mg, 2.80 mmol) was added. The resulting mixture was stirred at room temperature for 22.5 hours. Sodium bicarbonate (5% aq.) was added to quench the reaction. The organic phase was separated and the aqueous phase was extracted with dichloromethane (2×15 mL). The combined organic layers were washed with brine, dried over sodium sulfate, filtered, and the solvent removed under reduced pressure. The residue was purified by flash column chromatography (Biotage, reverse phase column, 20-50% MeCN/water in 25 CV) to afford N-mPEG3-C₃-mexiletine 3 (112 mg). ¹H-NMR (CDCl₃): 6.99-6.87 (m, 3H), 3.72-3.50 (m, 16H), 3.35 (s, 3H), 3.17-3.11 (m, 1H), 2.92-2.84 (m, 1H), 2.80-2.71 (m, 1H), 2.26 (s, 6H), 1.89-1.80 (m, 2H), 1.17 (d, J=6.3 Hz, 2H). LC-MS: 384.3 (MH⁺).

Synthesis of N-mPEG8-mexiletine 4 (n=8):

A reaction mixture of mexiletine hydrochloride (225 mg, 1.043 mmol), mPEG8-Br (538 mg, 1.203 mmol) and potassium carbonate (326 mg, 2.335 mmol) in water (3 mL) was placed in single-mode focused microwave reactor (CEM) and operated at 120° C. for 30 minutes. The mixture was diluted with water (3 mL) and extracted with EtOAc (3×15 mL). The combined organic layers were washed with brine, dried over Na₂SO₄, filtered, and the solvent removed under reduced pressure. The residue was purified by reverse phase column chromatography to afford the desired product, N-mPEG₈-mexiletine (260 mg) as an oil in 46% yield. ¹H-NMR (CDCl₃): 6.99-6.86 (m, 3H), 3.68-3.60 (m, 30H), 3.55-3.51 (m, 2H), 3.36 (s, 3H), 3.17-3.11 (m, 1H), 2.95-2.84 (m, 2H), 2.26 (s, 6H), 1.89-1.80 (m, 2H), 1.17 (d, J=6.3 Hz, 2H). LC-MS: 546.4 (MH⁺).

Synthesis of N-mPEG7-mexiletine 4 (n=7) and N,N-di-mPEG7-mexiletine 5 (n=7):

The same procedure for the synthesis of N-mPEG8-mexiletine was followed by using mexiletine hydrochloride (370 mg, 1.72 mmol), mPEG7-Br (1.402 g, 3.476 mmol) and potassium carbonate (710 mg, 5.09 mmol) in water (3 mL) to afford the product N-mPEG₇-mexiletine (205 mg) as an oil in 24% isolated yield and N,N-di-mPEG7-mexiletine (305 mg) as an oil in 22% isolated yield.

N-mPEG7-mexiletine 4 (n=7): ¹H-NMR (CDCl₃): 6.99-6.86 (m, 3H), 3.67-3.60 (m, 26H), 3.55-3.51 (m, 2H), 3.35 (s, 3H), 3.17-3.11 (m, 1H), 2.95-2.84 (m, 2H), 2.26 (s, 6H), 1.18 (d, J=6.3 Hz, 2H). LC-MS: 502.3 (MH⁺).

N,N-mPEG7-mexiletine 5 (n=7): ¹H-NMR (CDCl₃): 6.98-6.87 (m, 3H), 3.79-3.74 (m, 1H), 3.63-3.59 (m, 44H), 3.56-3.51 (m, 5H), 3.47 (t, J=6.9H, 4H), 3.36 (s, 6H), 3.23-3.13 (m, 1H), 2.78 (t, J=6.9H, 4H), 2.25 (s, 6H), 1.18 (d, J=6.3 Hz, 2H). LC-MS: 824.5 (MH⁺).

Synthesis of N-mPEG5-mexiletine 4 (n=5):

The same procedure for the synthesis of N-mPEG8-mexiletine was followed by using mexiletine hydrochloride (395 mg, 1.83 mmol), mPEG5-Br (710 mg, 2.25 mmol) and potassium carbonate (682 mg, 4.89 mmol) in water (5 mL) to afford the product N-mPEG5-mexiletine 4 (303 mg) as an oil in 40% isolated yield. ¹H-NMR (CDCl₃): 6.99-6.85 (m, 3H), 3.70-3.57 (m, 18H), 3.53-3.49 (m, 2H), 3.34 (s, 3H), 3.14-3.08 (m, 1H), 2.94-2.83 (m, 2H), 2.46 (br, NH, 1H), 2.25 (s, 6H), 1.15 (d, J=6.3 Hz, 2H). LC-MS: 414.3 (MH⁺).

Synthesis of N,N-di-mPEG5-mexiletine 5 (n=5):

A reaction mixture of mexiletine hydrochloride (431 mg, 2.0 mmol), mPEG5-Br (2.055 g, 6.52 mmol) and potassium carbonate (1.265 g, 9.06 mmol) in water (2 mL) was placed in single-mode focused microwave reactor (CEM) and operated at 120° C. for 40 minutes, 150° C. for 20 minutes. After a period of time, more mPEG₅-Br (358 mg, 1.136 mmol) was added. The mixture was irradiated using microwave conditions of 120° C. for another 40 minutes. The mixture was diluted with water (3 mL) and extracted with EtOAc (3×10 mL). The combined organic layers were washed with brine, dried over Na₂SO₄, filtered and the solvent removed under reduced pressure. The residue was purified by reverse phase column chromatography to afford the desired product N,N-di-mPEG5-mexiletine 5 (973 mg) as an oil in 75% isolated yield. ¹H-NMR (CDCl₃): 6.98-6.85 (m, 3H), 3.79-3.74 (m, 1H), 3.63-3.55 (m, 28H), 3.53-3.51 (m, 5H), 3.47 (t, J=6.9H, 4H), 3.36 (s, 6H), 3.23-3.18 (m, 1H), 2.78 (t, J=6.9H, 4H), 2.24 (s, 6H), 1.19 (d, J=6.3 Hz, 2H). LC-MS: 648.4 (MH⁺).

Synthesis of N-mPEG3-mexiletine 4 (n=3) and N,N-di-mPEG3-mexiletine 5 (n=3):

The same procedure for the synthesis of N-mPEG8-mexiletine was followed by using mexiletine hydrochloride (294 mg, 1.36 mmol), mPEG3-Br (658 mg, 2.90 mmol) and potassium carbonate (614 mg, 4.40 mmol) in water (2 mL) to afford the product N-mPEG3-mexiletine 4 (191 mg) as an oil in 43% isolated yield and N, N-di-mPEG₃-mexiletine 5 (65 mg) as an oil in 10% isolated yield.

N-mPEG3-mexiletine 4 (n=3): ¹H-NMR (CDCl₃): 6.99-6.87 (m, 3H), 3.72-3.61 (m, 10H), 3.52-3.49 (m, 2H), 3.33 (s, 3H), 3.17-3.11 (m, 1H), 2.97-2.83 (m, 2H), 2.25 (s, 6H), 1.18 (d, J=6.3 Hz, 2H). LC-MS: 326.3 (MH⁺).

N,N-di-mPEG₃-mexiletine 5 (n=3): ¹H-NMR (CDCl₃): 6.99-6.85 (m, 3H), 3.80-3.63 (m, 1H), 3.62-3.55 (m, 12H), 3.54-3.50 (m, 5H), 3.48 (t, J=6.9H, 4H), 3.35 (s, 6H), 3.23-3.17 (m, 1H), 2.79 (t, J=6.9H, 4H), 2.25 (s, 6H), 1.18 (d, J=6.6 Hz, 2H). LC-MS: 472.3 (MH⁺).

Synthesis of PEG4-di-OMs 7:

Methanesulfonyl chloride (6 mL, 76.82 mmol) was added dropwise to a stirred solution of PEG4-di-OH 6 (4.455 g, 22.59 mmol) and TEA (15 mL, 107.08 mmol) in dichloromethane (20 mL) at 0° C. After the addition was complete, the resulting mixture was stirred at room temperature for one day. Water was added to quench the reaction. The organic phase was separated and the aqueous phase was extracted with dichloromethane (2×50 mL). The combined organic layers were washed with brine (3×60 mL), dried over Na₂SO₄, filtered, and the solvent removed under reduced pressure to afford the desired product (7.7347 g) in 98% yield. ¹H-NMR (CDCl₃): 4.37-4.34 (m, 4H), 3.76-3.73 (m, 4H), 3.67-3.60 (m, 8H), 3.05 (s, 6H).

Synthesis of PEG4-di-Mexiletine 8:

The same procedure for the synthesis of N-mPEG8-mexiletine was followed by using mexiletine hydrochloride (474 mg, 2.2 mmol), PEG4-di-OMs 7 (388 mg, 1.1 mmol) and potassium carbonate (1.289 g, 9.23 mmol) in water (2 mL) to afford the product PEG4-di-mexiletine 8 (47 mg). ¹H-NMR (CDCl₃): 6.98-6.86 (m, 6H), 3.70-3.57 (m, 16H), 3.14-3.08 (m, 1H), 2.94-2.80 (m, 2H), 2.64 (br, NH, 2H), 2.25 (s, 12H), 1.15 (d, J=6.3 Hz, 4H). LC-MS: 517.4 (MH⁺).

Various pKa and Log P for values for various conjugates prepared in this example were determined and are reported in Table 2.

TABLE 2
pKa and LogP for Various Mexiletine Conjugates

| Samples | pKa₁ | pKa₂ | LogP |
|---|---|---|---|
| Mexiletine hydrochloride | 9.14 | | 2.47 |
| N,N-di-mPEG5-Mexilentine | 8.26 | | 1.61 |
| N-mPEG5-Mexiletine | 8.27 | 7.86 | 4.51 |
| PEG4-di-Mexiletine | 7.73 | | 2.36 |
| N-mPEG3-Mexiletine | 8.27 | | 2.26 |
| N-mPEG3-C₃-Mexiletine | 8.98 | | 2.05 |
| N-mPEG8-Mexiletine | 8.79 | | 1.79 |
| N,N-di-mPEG7-Mexiletine | 7.75 | | 0.83 |
| N-mPEG7-Mexiletine | 8.26 | | 1.23 |
| N,N-di-mPEG3-Mexiletine | 7.76 | | 1.96 |

### Example 3

### Potency at Sodium Channels

Potency of the PEG lidocaine and PEG mexilitine conjugates were measured in isolated human atrial cells using electrophysiological techniques. Briefly, atrial cardiomyocytes were isolated from human atrial appendages using standard collagenase digestion techniques [Crumb et al. (1995) Am J Physiol 268:H1335-H1342]. For the study of the sodium current in human myocytes, cells were perfused with an external solution that consists of (in mmol/L): 115 TMA chloride, 10 NaCl, 5 CsCl, 1.8 CaCl₂), 1.2 MgCl₂, 10 HEPES, 11 dextrose, pH adjusted to 7.4 with TMA-OH. The chemical composition of the internal solution used was (in mM): 115 CsF, 20 CsCl, 10 NaF, 10 HEPES, 5 EGTA; pH adjusted to 7.2 with CsOH. Experiments were performed at 23±1° C. Currents were measured using the whole-cell variant of the patch clamp method. An Axopatch 1-B amplifier (Axon Instruments, Foster City, Calif.) was used for whole-cell voltage clamping. After rupture of the cell membrane (entering whole-cell mode), current kinetics and amplitudes were allowed to stabilize as the cell was dialyzed with internal solution and paced at 1 Hz (typically 3-5 minutes). Currents were considered stable if currents elicited by a series of voltage pulses given at 0.1 Hz were superimposed. I_{Na} recorded from human atrial myocytes was elicited by a pulse to −20 mV from a holding potential of −120 mV (pulse duration is 40 ms). Peak inward current was measured for I_{Na}. Pacing rates of 0.1 and 3 Hz were examined. Creation of voltage clamp pulses and data acquisition were controlled by a PC running pClamp software (version 9.2, Axon Instruments). Test compounds were added in a cumulative manner.

Data are presented as % reduction of current amplitude. This was measured as current reduction after a steady-state effect had been reached in the presence of drug relative to current amplitude before drug was introduced (control). Each cell served as its own control. Log-linear plots were created of the mean percent blockade ±SEM at the concentrations tested. Nonlinear curve fitting routine was utilized to fit a three-parameter Hill equation to the results using MicroCal Origin, version 6.0 software. The equation is:

$$y = {V}_{max} \frac{{x}^{n}}{{k}^{n} + {x}^{n}}$$

where Vₘₐₓ, k, and n are unconstrained variables (except Vₘₐₓ>0). Since the Vₘₐₓ parameter was not constrained to 100%, the parameter k does not represent an IC₅₀ for ion channel blockade. Thus, the IC₅₀ was calculated from the inverse of the previous equation:

$$x = {k ( \frac{y}{{V}_{max} - y} )}^{1 / n}$$

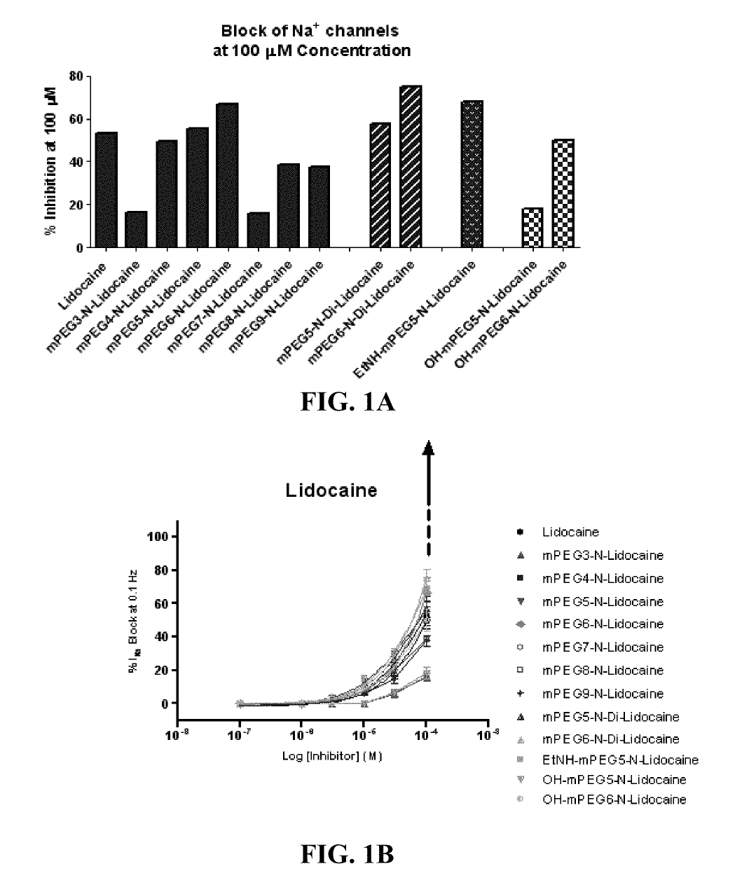

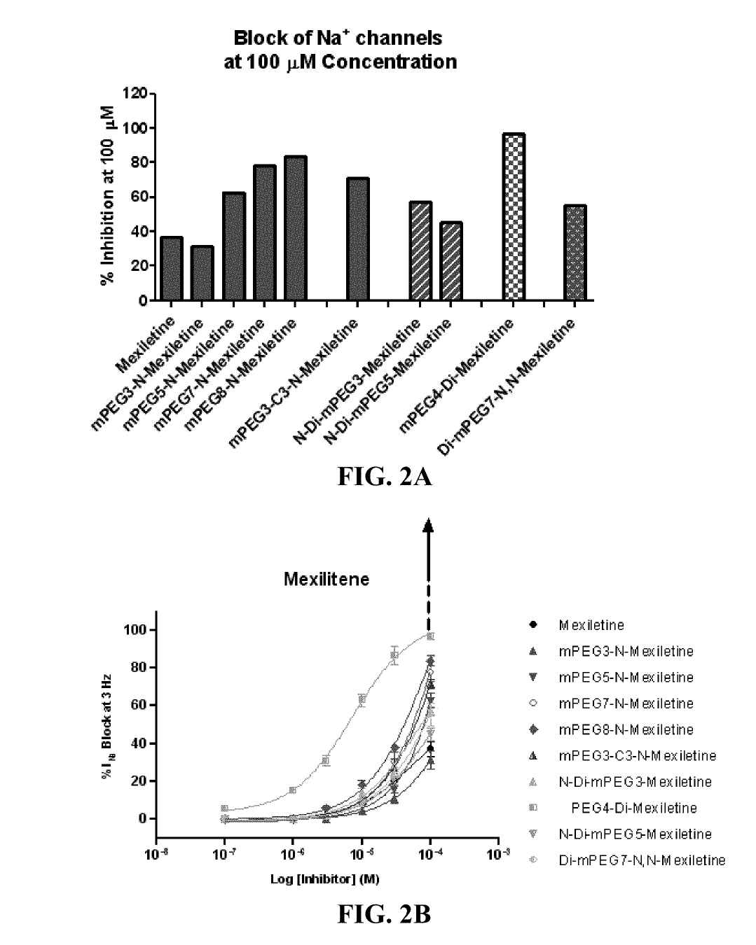

The data show (Tables 3 and 4 and FIG. 1A, FIG. 1B, FIG. 2A and FIG. 2B) that the PEG conjugates of lidocaine and mexilitine retain activity in vitro and inhibit human Na channels expressed in native cells with IC₅₀ values that are comparable to that of the parent molecules.

TABLE 3
Results of Potency at Sodium Channels Assays—“Lidocaine Series”

| Compound | IC₅₀ at 0.1 Hz (M) | Fold Change vs. Parent |
|---|---|---|
| Lidocaine | 9.88E−05 | 1 |
| mPEG3-Lidocaine | 7.06E−04 | 7 |
| mPEG4-Lidocaine | 1.78E−04 | 2 |
| mPEG5-Lidocaine | 5.35E−05 | 1 |
| mPEG6-Lidocaine | 4.73E−04 | 5 |
| mPEG7-Lidocaine | 2.36E−04 | 2 |
| mPEG8-Lidocaine | 5.75E−05 | 1 |
| mPEG9-Lidocaine | 1.73E−04 | 2 |
| mPEG5-Di-Lidocaine | 8.33E−05 | 1 |
| mPEG6-Di-Lidocaine | 2.36E−04 | 2 |
| EtNH-PEG5-Lidocaine | 9.93E−05 | 1 |
| PEG5-Lidocaine | 3.30E−04 | 3 |
| PEG6-Lidocaine | 1.04E−04 | 1 |

TABLE 4
Results of Potency at Sodium Channels Assays—“Mexiletine Series”

| Compound | IC₅₀ at 0.1 Hz (M) | Fold Change vs. Parent |
|---|---|---|
| Mexiletine | 5.36E−05 | 1 |
| N-mPEG3-Mexiletine | 2.84E−04 | 5 |
| N-mPEG5-Mexiletine | ~5.49E−01 | ~10243 (Ambiguous) |
| N-mPEG7-Mexiletine | 2.06E−04 | 4 |
| N-mPEG8-Mexiletine | 9.05E−05 | 2 |
| N-mPEG3-C₃-Mexiletine | 1.88E−04 | 4 |
| N,N-Di-mPEG3-Mexiletine | 6.62E−05 | 1 |
| PEG4-Di-Mexiletine | 7.32E−06 | 0.14 |
| N,N-Di-mPEG5-Mexiletine | 9.59E−05 | 2 |
| N,N-Di-mPEG7-Mexiletine | 1.30E−04 | 2 |

### Example 4

### Analgesic Assay

An analgesic assay was used to determine whether a given compound can reduce and/or prevent visceral pain in mice.

The assay utilized CB-1 male mice (5-8 mice per group), each mouse being approximately 0.015-0.030 kg on the study day. Mice were treated according to standard protocols.

Mice were given a single “pretreatment” dose of a compound lacking covalent attachment of a water-soluble, non-peptidic oligomer, a corresponding conjugate comprising the compound covalently attached to a water-soluble, non-peptidic oligomer, or control solution (IV, SC, IP or orally) thirty minutes prior to the administration of the acetic acid solution. The animal was given an IP injection of an irritant (acetic acid) that induces “writhing” which may include: contractions of the abdomen, twisting and turning of the trunk, arching of the back and the extension of the hind limbs. Mice were given a single IP injection (0.1 mL/10 g bodyweight) of a 0.5% acetic acid solution. After the injection the animals were returned to their cages and their behavior was observed. Contractions were counted between 0 and 20 minutes after the injection. The animals were used once. Each tested article was dosed at 1, 3 and 10 mg/kg (n=5 animals/dose). Table 3 shows the group assignments used for the assay.

TABLE 3
Group Assignments

| | | | Dose of | | Irritant |
|---|---|---|---|---|---|
| | | | Test | Dose | 0.5% |
| Group | | Route of | Article | Volume | Acetic |
| No. | Test Article | Administration | (mg/kg) | (mL) | Acid |
| 1 | Vehicle | SC | NA | 0.1 | 0.1 mL/10 |
| | Control Saline | | | | g BW |
| 2 | Morphine | SC | 0.5 | 0.1 | 0.1 mL/10 |
| | (Saline) | | | | g BW |
| 3 | Loperamide | SC | 0.4 | 0.1 | 0.1 mL/10 |
| | (Saline) | | | | g BW |
| 4 | Tested Article | SC | 1 | 0.1 | 0.1 mL/10 |
| | | | | | g BW |
| 5 | Tested Article | SC | 3 | 0.1 | 0.1 mL/10 |
| | | | | | g BW |
| 6 | Tested Article | SC | 10 | 0.1 | 0.1 mL/10 |
| | | | | | g BW |

A compound is considered to have analgesia activity if, upon administration of the compound, there is at least a ≥30% decrease in writhing in at least 40% of test animals. For comparison purposes, it is useful to compare the analgesia activity against the standard analgesic, morphine. The results of morphine and saline are provided in Table 4. As used herein, “Responders” equal the number of animal displaying≤writhes as upper 95% confidence limit for morphine and “responding faction” equals the number of responding animals/N.

TABLE 4
Saline and Morphine Results of Acetic Acid Writhing Test

| | | | | | # of | Responding | | % Reduction |
|---|---|---|---|---|---|---|---|---|
| | Mean | SEM | CV | N | Responders | Fraction | ± | in Writhing |
| Historical | 49.08 | 3.15 | 0.064 | 25 | 1 | 0.04 | 0.003 | 0% |
| Saline | | | | | | | | |
| Historical | 21.33 | 3.41 | 0.160 | 27 | 15 | 0.56 | 0.089 | 57% |
| Morphine | | | | | | | | |

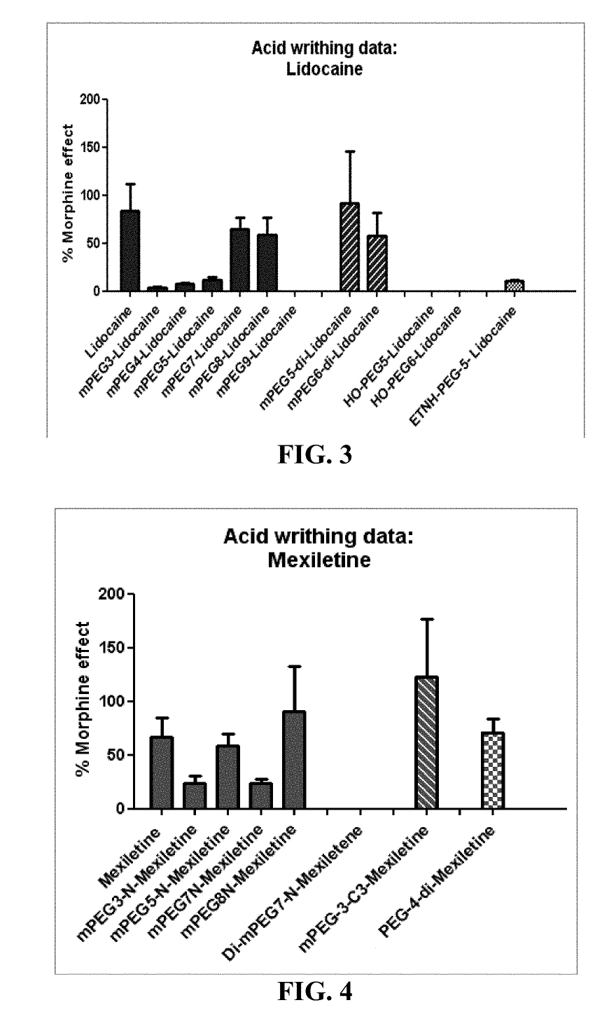

The results of the most potent responders are provided in Table 5 and provided in graph form in FIG. 3 for lidocaine and conjugates thereof and FIG. 4 for mexiletine and conjugates thereof.

TABLE 5
Most Potent Responders

| Compound | | | | % Reduction | # of | Responding |
|---|---|---|---|---|---|---|
| (dose) | Mean | SEM | N | in Writhing | Responders | Fraction |
| Loperamide | 9.0 | 5.2 | 4 | 78.6 | 4 | 1.00 |
| (0.75 mg/kg) | | | | | | |
| Lidocaine | 36.3 | 12.3 | 4 | 37.5 | 2 | 0.50 |
| (10 mg/kg) | | | | | | |
| mPEG5-N-Mexilitene | 29.0 | 5.6 | 5 | 42.4 | 2 | 0.40 |
| (10 mg/kg) | | | | | | |
| mPEG8-N-Mexilitene | 17.2 | 8.1 | 5 | 65.8 | 3 | 0.60 |
| (10 mg/kg) | | | | | | |
| mPEG5-di-Lidocaine | 17.3 | 10.0 | 4 | 60.3* | 2 | 0.50* |
| (10 mg/kg) | | | | | | |
| PEG6-di-Lidocaine | 27.0 | 10.9 | 5 | 37.9 | 3 | 0.60 |
| (10 mg/kg) | | | | | | |
| mPEG5-di-Lidocaine | 23.5 | 10.5 | 4 | 46.0 | 2 | 0.50 |
| (3 mg/kg) | | | | | | |
| mPEG3-C3-Mexiletine | 29.8 | 13.2 | 5 | 46.3 | 2 | 0.40 |
| (10 mg/kg) | | | | | | |
| mPEG7- Lidocaine | 36.0 | 7.2 | 5 | 40.5 | 2 | 0.40 |
| (10 mg/kg) | | | | | | |
| mPEG8- Lidocaine | 38.0 | 11.4 | 5 | 37.2 | 2 | 0.40 |
| (10 mg/kg) | | | | | | |

Other results (not shown) may not have been active according to the definition used herein, but even a relatively low response may be remedied by administration of an increased dose to the patient.

What is claimed is:

1. A compound comprising a lidocaine residue covalently attached via a linkage to a water-soluble, non-peptidic oligomer.

2. The compound of claim 1, wherein the linkage is a stable linkage.

3. The compound of claim 1, wherein the linkage is a degradable linkage.

4. The compound of claim 1, wherein the linkage is an amine linkage.

5. The compound as in any one of claims 1, 2, 3 and 4, wherein the weight average molecular weight of the water-soluble, non-peptidic oligomer is less than 400 Daltons.

6. The compound of claim 1, having the following structure: 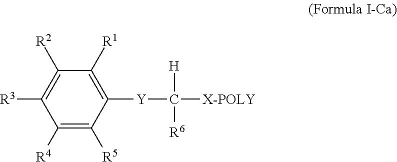
wherein:
R¹ is hydrogen or lower alkyl;
R² is hydrogen or lower alkyl;
R³ is hydrogen or lower alkyl;
R⁴ is hydrogen or lower alkyl;
R⁵ is hydrogen or lower alkyl;
R⁶ is hydrogen or lower alkyl;
Y is selected from the group consisting of —O—, —O—CH₂—, —CH₂—O—, —NH—, —NHC(O)—, —C(O)NH—, —CH₂— and —CH₂—CH₂—;
X is a spacer moiety; and
POLY is a water-soluble, non-peptidic oligomer.

7. The compound of claim 1, having the following structure: 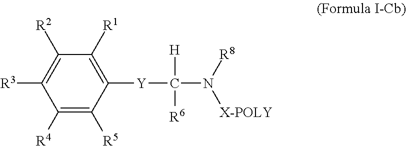
wherein:
R¹ is hydrogen or lower alkyl;
R² is hydrogen or lower alkyl;
R³ is hydrogen or lower alkyl;
R⁴ is hydrogen or lower alkyl;
R⁵ is hydrogen or lower alkyl;
R⁶ is hydrogen or lower alkyl;
R⁸ is hydrogen or lower alkyl;
Y is selected from the group consisting of —O—, —O—CH₂—, —CH₂—O—, —NH—, —NHC(O)—, —C(O)NH—, —CH₂— and —CH₂—CH₂—;
X is a spacer moiety; and
POLY is a water-soluble, non-peptidic oligomer.

8. The compound as in any one of claims 1, 2, 3 and 4, wherein the lidocaine residue is a residue of a compound having the following structure: 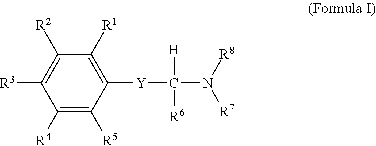
wherein:
R¹ is hydrogen or lower alkyl;
R² is hydrogen or lower alkyl;
R³ is hydrogen or lower alkyl;
R⁴ is hydrogen or lower alkyl;
R⁵ is hydrogen or lower alkyl;
R⁶ is hydrogen or lower alkyl;
R⁷ is hydrogen or lower alkyl;
R⁸ is hydrogen or lower alkyl; and
Y is selected from the group consisting of —O—, —O—CH₂—, —CH₂—O—, —NH—, —NHC(O)—, —C(O)NH—, —CH₂— and —CH₂—CH₂—.

9. The compound as in any one of claims 1, 2, 3 and 4, wherein the lidocaine residue is a residue of a Class IB antiarrhythmic drug.

10. The compound as in any one of claims 1, 2, 3 and 4, wherein the lidocaine residue is a residue of lidocaine.

11. The compound as in any one of claims 1, 2, 3 and 4, wherein the lidocaine residue is a residue of tocainide.

12. The compound as in any one of claims 1, 2, 3 and 4, wherein the lidocaine residue is a residue of mexiletine.

13. The compound as in any one of claims 1, 2, 3, 4, 5, 6, 7, 8, 9, 10, 11 and 12, wherein the water-soluble, non-peptidic oligomer is a poly(alkylene oxide).

14. The compound of claim 13, wherein the poly(alkylene oxide) is a poly(ethylene oxide).

15. The compound of claim 1, wherein the water-soluble, non-peptidic oligomer has a number of repeating monomers in the range of from 1 to 30.

16. The compound of claim 15, wherein the water-soluble, non-peptidic oligomer has a number of repeating monomers in the range of from 1 to 10.

17. The compound of claim 13, wherein the poly(alkylene oxide) includes an alkoxy or hydroxy end-capping moiety.

18. The compound of claim 1, wherein the compound has a single lidocaine residue covalently attached to a single water-soluble, non-peptidic oligomer.

19. The compound of claim 1, wherein the compound has a single lidocaine residue covalently attached to two water-soluble, non-peptidic oligomers.

20. The compound of claim 1, wherein the compound has two lidocaine residues covalently attached to a single water-soluble, non-peptidic oligomer.

21. The compound of claim 1, having the following structure: 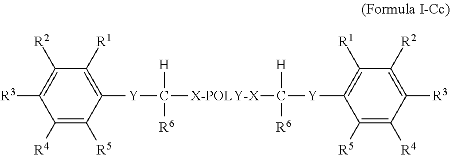
wherein:
each R¹ is hydrogen or lower alkyl;
each R² is hydrogen or lower alkyl;
each R³ is hydrogen or lower alkyl;
each R⁴ is hydrogen or lower alkyl;
each R⁵ is hydrogen or lower alkyl;
each R⁶ is hydrogen or lower alkyl;
each Y is selected from the group consisting of —O—, —O—CH₂—, —CH₂—O—, —NH—, —NHC(O)—, —C(O)NH—, —CH₂— and —CH₂—CH₂—;
each X is a spacer moiety; and
POLY is a water-soluble, non-peptidic oligomer.

22. The compound of claim 1, having the following structure: 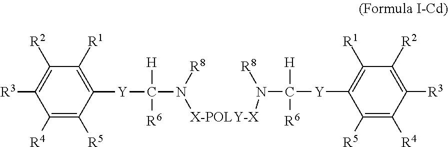
wherein:
each R¹ is hydrogen or lower alkyl;
each R² is hydrogen or lower alkyl;
each R³ is hydrogen or lower alkyl;
each R⁴ is hydrogen or lower alkyl;
each R⁵ is hydrogen or lower alkyl;
each R⁶ is hydrogen or lower alkyl;
each R⁸ is hydrogen or lower alkyl;
each Y is selected from the group consisting of —O—, —O—CH₂—, —CH₂—O—, —NH—, —NHC(O)—, —C(O)NH—, —CH₂— and —CH₂—CH₂—;
each X is a spacer moiety; and
POLY is a water-soluble, non-peptidic oligomer.

23. The compound of claim 1, having the following structure: 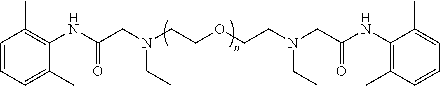
wherein (n) is an integer having a value in the range of from 1 to 30.

24. The compound of claim 1, having the following structure: 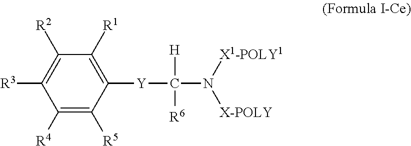
wherein:
R¹ is hydrogen or lower alkyl
R² is hydrogen or lower alkyl;
R³ is hydrogen or lower alkyl;
R⁴ is hydrogen or lower alkyl;
R⁵ is hydrogen or lower alkyl;
R⁶ is hydrogen or lower alkyl;
R⁸ is hydrogen or lower alkyl;
Y is selected from the group consisting of —O—, —O—CH₂—, —CH₂—O—, —NH—, —NHC(O)—, —C(O)NH—, —CH₂— and —CH₂—CH₂—;
X is a spacer moiety;
X¹ is a spacer moiety;
POLY is a water-soluble, non-peptidic oligomer; and
POLY¹ is a spacer moiety.

25. The compound of claim 1, having the following structure: 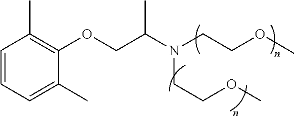
wherein (n) is an integer in the range of from 1 to 30.

26. The compound of claim 1, having the following structure: 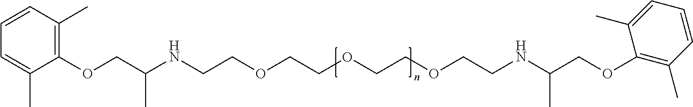
wherein (n) is in the range of 1 to 28.

27. A composition comprising a compound comprising (i) a lidocaine residue covalently attached via a linkage to a water-soluble, non-peptidic oligomer, and (ii) optionally, a pharmaceutically acceptable excipient.

28. A composition of matter comprising a compound comprising a lidocaine residue covalently attached via a linkage to a water-soluble, non-peptidic oligomer, wherein the compound is present in a dosage form.

29. A method comprising covalently attaching a water-soluble, non-peptidic oligomer to a lidocaine residue.

30. A method comprising administering to a subject a compound comprising a lidocaine residue covalently attached via a linkage to a water-soluble, non-peptidic oligomer.
